(19) United States
(12) Patent Application Publication
(10) Pub. No.: US 2008/0132919 A1
(43) Pub. Date: Jun. 5, 2008

(54) Cycling suturing and knot-tying device

(76) Inventors: Faising Chui, Santa Barbara, CA (US); Claude Vidal, Santa Barbara, CA (US); Russell J. Redmond, Goleta, CA (US); Michael Collinson, Goleta, CA (US)

Correspondence Address: JAMES E. BRUNTON, ESQ., P. O. BOX 29000, GLENDALE, CA, 91209, US

(21) Appl. No.: 12/012,816

(22) Filed: Feb. 5, 2008

Related U.S. Application Data
(63) Continuation-in-part of application No. 11/223,737, filed on Sep. 9, 2005, now Pat. No. 7,338,504.
Application No. 11/223,737 is a continuation-in-part of application No. 10/263,902, filed on Oct. 3, 2002, now Pat. No. 7,004,951.

(51) Int. Cl. A61B 17/04

(52) U.S. Cl. 606/145

(57) ABSTRACT

A cycling, suturing and knot-tying device is characterized by an arcuate fixed, grooved or recessed way provided in a correspondingly shaped support frame for accommodating a curved needle fitted with thread, and frictional needle-engaging devices provided in the way for selectively engaging the needle and driving the needle in one or both rotational directions to suture a wound with the thread. Selective articulation of the frame and the way and driving of the needle in the way by positioning the frictional needle-engaging devices with respect to the needle are typically effected by manipulation of a pistol-grip operating apparatus having a transmission tube that mounts the frame and the way in articulating relationship and rotates and articulates with respect to the pistol grip and carries various operating elements that interface with the frictional needle-engaging devices in the way. In at least one embodiment needle direction-adjusting elements are provided in the frame in cooperation with selected devices in the way for determining the direction of needle rotation responsive to manipulation of an interfacing operating element located on the operating apparatus. Auxiliary thread-handling or incrementing and knot-tying devices are also disclosed.

This is a Continuation-In-Part of co-pending application Ser. No. 11/223,737 filed Sep. 9, 2005 which is a Continuation-In-Part of 10/263,902 filed Oct. 3, 2002, now U.S. Pat. No. 7,004,951 issued Feb. 28, 2006.

### BACKGROUND OF THE INVENTION

### Field of the Invention

This invention relates to the suturing of surgical incisions, wounds and any other joining or fixing of tissue in general surgical procedures and, in particular, the suturing, joining or binding of tissue in surgical procedures involving very small, cramped or otherwise inaccessible fields of operation, such as, in general, laparoscopic and neurological brain surgery. One of the problems which is inherent in many surgical procedures is that of limited access of the surgeon's hands, as well as the needle and suturing implements, into the incision or wound. This problem is amplified under circumstances where delicate surgery such as heart, brain, and spinal surgery, as well as surgery on infants and children is undertaken, since the surgical areas of interest involve minute features. Many surgical procedures that would otherwise be possible on adults and children are impossible due to the tiny operating fields, and many conditions that might otherwise be corrected by surgery are, therefore, considered to be inoperable. The same situation occurs under circumstances such as suturing within interior and normally inaccessible areas of the body where no known surgical techniques and/or instruments can access these areas and provide the necessary surgical relief.

The micro-sized cyclical suturing and knot-tying device of this invention is designed to optimize surgical suturing and, in particular, to facilitate access to very small, normally, but not limited to, inaccessible areas of the body, including the heart, brain and spinal cord, as well as conventional procedures on infants, to allow surgical relief which has heretofore been unavailable by conventional surgical techniques. The device of this invention is characterized by an arcuate, fixed, grooved or recessed way provided in a correspondingly configured support frame capable of receiving a curved needle fitted with a length of thread. The curved or arcuate frame is constructed to support the needle from end to end when the needle is in the starting position. The arcuate, grooved or recessed support-way is shaped in such a manner as to provide the correspondingly shaped needle with adequate support while leaving the top of the groove or recess, located on the top of the disk of rotation, open to permit passage of the thread around the way circuit traversed by the needle without trapping the thread in any of the needle support and drive structure. Both the needle and the frame, as well as the way, have a corresponding gap to accommodate tissue to be sewn. Accordingly, when the needle is driven in a circular path by manipulation of the appropriate operating components or elements in the operating device, the needle passes through tissue which protrudes into the gap in the way. Furthermore, since the thread is attached to the needle, the thread is drawn behind the needle, around the open top of the way, across the gap and through the tissue as the needle traverses the tissue. Although the thread cannot be trapped within the way, it is entrapped within the tissue through which the needle and thread is passed.

Various elements and components are provided in the operating device in cooperation with the way, the needle and the support frame for effecting rotation of the needle in response to manipulation of the operating device.

The device also uniquely includes means that allows the suturing head to be controllably moved arcuately upwardly and downwardly relative to the hollow-barrel portion of the device to which it is connected. Additionally, means are provided for controllably rotating the hollow-barrel portion of the device relative to the hand grip portion of the device to which it is connected.

### SUMMARY OF THE INVENTION

The foregoing and other objects of the invention are provided in a suturing device which includes an arcuate frame having an open groove; an arcuate needle disposed within the frame and seated in the groove; engaging means disposed within the frame and selectively extending into the groove for releasably engaging the needle; and drive means engaging the engaging means for driving the needle in the groove.

One object of one form of the invention is to provide a suturing device which comprises a uniquely configured articulating, suturing head that includes a plurality of strategically shaped, circumferentially spaced cavities, a generally semi-circular-shaped shuttle track along which a novel needle advancing shuttle is slidably movable and a generally semi-circular-shaped needle guide along which a novel suturing needle is sequentially advanced by movement of the needle advancing shuttle.

Another object of one form of the invention is to provide a suturing device of the character described in the preceding paragraph which includes a suturing needle that has a novel rectangular cross section, a circumferentially extending, notched wall and a strategically angled, chamfered end that compensates for needle deformation in the suturing process.

Another object of one form of the invention is to provide a suturing device of the aforementioned character, which includes a plurality of uniquely configured needle-engaging and advancing members that are disposed within the plurality of strategically shaped, circumferentially spaced cavities formed in the articulating, suturing head portion and are adapted for both transverse and pivotal movement within the cavities in response to movement of the needle advancing shuttle.

Another object of one form of the invention is to provide a suturing device of the character described in the preceding paragraphs which comprises a dual-cable shuttle advancing subsystem that includes a cooperating biasing spring and trigger mechanism for smoothly and positively moving the needle advancing shuttle along the shuttle track of the suture head of the device.

Another object of the invention is to provide means that allows the suturing head of the device to be controllably moved arcuately upwardly and downwardly relative to the hollow-barrel portion of the device to which it is connected.

Another object of the invention is to provide means for controllably rotating the hollow-barrel portion of the device relative to the hand grip portion of the device to which it is connected.

These and other objects of the invention will be achieved by the novel apparatus of the invention, the details of which are discussed in the paragraphs that follow.

### BRIEF DESCRIPTION OF THE DRAWINGS

FIG. 1 is a generally perspective view of one form of the suturing device of the present invention.

FIG. 2 is a generally perspective view of the suturing device shown in FIG. 1 as viewed from one side of the device and broken-away to show internal construction.

FIG. 3 is a greatly enlarged, generally perspective, fragmentary view of the area designated in FIG. 2 as “3”.

FIG. 4 is a generally perspective view, similar to FIG. 2, but showing the trigger in an actuated position.

FIG. 5 is a greatly enlarged, generally perspective, fragmentary view of the area designated in FIG. 4 as “5”.

FIG. 6 is a generally perspective view of the suturing device shown in FIG. 1 as viewed from the opposite side of the device and broken-away to show internal construction.

FIG. 7 is a greatly enlarged, generally perspective, fragmentary view of the area designated in FIG. 6 as “7”.

FIG. 8 is a view similar to FIG. 6, but showing the trigger in an actuated position.

FIG. 9 is a greatly enlarged, generally perspective, fragmentary view of the area designated in FIG. 8 as “9”.

FIG. 10 is a greatly enlarged, generally perspective, fragmentary view of the articulable head portion of the device and of the coupling sub-assembly for coupling the head portion to the barrel portion of the device.

FIG. 11 is a top plan view of the articulable head portion of the device as it appears after the cover portions have been removed to reveal the internal construction thereof.

FIG. 12 is a top plan view similar to FIG. 11, but showing the location of the suturing needle of the device after it has been moved from the position illustrated in FIG. 11 to a first advanced position.

FIG. 13 is a generally perspective, top view similar to FIG. 12 further showing the location of the suturing needle of the device after it has been advanced in a clockwise direction.

FIG. 14 is a generally perspective, exploded view showing more clearly the various operating components of the head portion of the suturing device.

FIG. 15 is a greatly enlarged, generally perspective view of one form of the suturing needle of this latest form of the suturing device.

FIG. 16 is a greatly enlarged, generally perspective view of one of the needle-engaging members of the invention that, during operation of the device, function to control movement of the suturing needle within a suturing needle guide-way formed in the body of the head portion of the device.

FIG. 17 is a greatly enlarged, diagrammatic view of the needle-engaging members of the invention illustrating their interaction with the needle during advancement of the shuttle member.

FIG. 18 is a greatly enlarged, diagrammatic view of the needle-engaging members of the invention illustrating their interaction with the needle during retraction of the shuttle member.

FIG. 19 is a generally enlarged, diagrammatic view of the head portion of the apparatus broken-away to illustrate the cooperative interaction of the operating cables of the apparatus on the shuttle member.

FIG. 20 is a generally perspective view of an alternate form of the suturing device of the present invention.

FIG. 21 is a generally perspective view of the suturing device shown in FIG. 20 as viewed from one side of the device and broken-away to show internal construction.

FIG. 22 is a side-elevational view of the suturing device shown in FIG. 20 as viewed from one side of the device and shown partly in cross section.

FIG. 23 is a greatly enlarged, cross-sectional view of the area designated in FIG. 22 as “23”.

FIG. 24 is a greatly enlarged, cross-sectional view of the area designated in FIG. 22 as “24”.

FIG. 25 is a top plan view of the articulable head portion of this alternate form of the device as it appears after the cover portions have been removed to reveal the internal construction thereof.

FIG. 26 is a greatly enlarged, generally perspective view of one form of the suturing needle of this latest form of the suturing device.

FIG. 27 is a greatly enlarged, side-elevational view of the suturing needle of this latest form of the suturing device.

FIG. 28 is a greatly enlarged, top plan view of the suturing needle of this latest form of the suturing device.

FIG. 29 is a top plan view similar to FIG. 25, but showing the location of the suturing needle of the device after it has been moved from the position illustrated in FIG. 25 to a first advanced position.

FIG. 30 is a greatly enlarged view of the area designated in FIG. 29 as “30”.

FIG. 31 is a top plan view similar to FIG. 29, but showing the needle driving member in a release position.

FIG. 32 is a greatly enlarged view of the area designated in FIG. 31 as “32”.

FIG. 33 is a top plan view of an alternate form of the articulable head portion of this alternate form of the device as it appears after the cover portions have been removed to reveal the internal construction thereof.

FIG. 34 is a top plan view similar to FIG. 33, but showing the location of the operating cables of the device.

FIG. 35 is a greatly enlarged view of the area designated in FIG. 34 as “35”.

FIG. 36 is a top plan view similar to FIG. 34, but showing the needle driving member of this latest form of the invention in a release position.

FIG. 37 is a greatly enlarged view of the area designated in FIG. 36 as “37”.

FIG. 38 is a top plan view similar to FIG. 36, but showing the location of the suturing needle of the device after it has been moved from the position illustrated in FIG. 36 to a second advanced position.

FIG. 39 is a top plan view of still another alternate form of the articulable head portion of this alternate form of the device as it appears after the cover portions have been removed to reveal the internal construction thereof.

FIG. 40 is a greatly enlarged view of the area designated in FIG. 39 as “40”.

FIG. 41 is a top plan view similar to FIG. 39, but showing the location of the suturing needle of the device after it has been moved from the position illustrated in FIG. 39 to an advanced position.

FIG. 42 is a greatly enlarged view of the area designated in FIG. 41 as “42”.

FIG. 43 is a greatly enlarged, side-elevational view of one form of the suturing head of the apparatus of the invention.

FIG. 44 is a greatly enlarged view of the area designated in FIG. 43 as “44”.

FIG. 45 is a generally perspective view of the suturing device shown in FIG. 20 as viewed from one side of the device and broken-away to show internal construction.

FIG. 46 is a greatly enlarged view of the area designated in FIG. 45 as “46”.

FIG. 47 is a greatly enlarged view of the area designated in FIG. 45 as “47”.

FIG. 48 is a generally perspective view of the suturing device similar to FIG. 45, but showing the suture head moved angularly upward relative to the barrel.

FIG. 49 is a greatly enlarged view of the area designated in FIG. 48 as “49”.

FIG. 50 is a greatly enlarged view of the area designated in FIG. 48 as “50”.

FIG. 51 is a generally perspective view of the suturing device also similar to FIG. 45, but showing the suture head moved angularly downward relative to the barrel.

FIG. 52 is a greatly enlarged view of the area designated in FIG. 51 as “52”.

FIG. 53 is a greatly enlarged view of the area designated in FIG. 51 as “53”.

FIG. 54 is an enlarged, generally perspective view showing the suture head moved angularly upward relative to the barrel connector.

FIG. 55 is an enlarged, generally perspective view showing the suture head moved angularly downward relative to the barrel connector.

### DESCRIPTION OF THE INVENTION

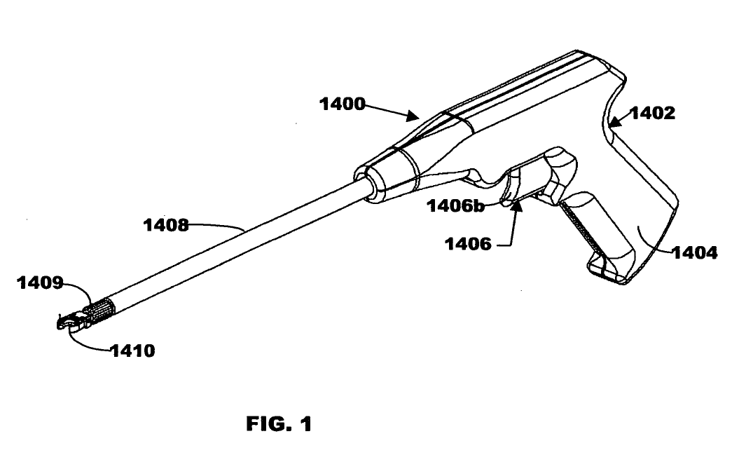

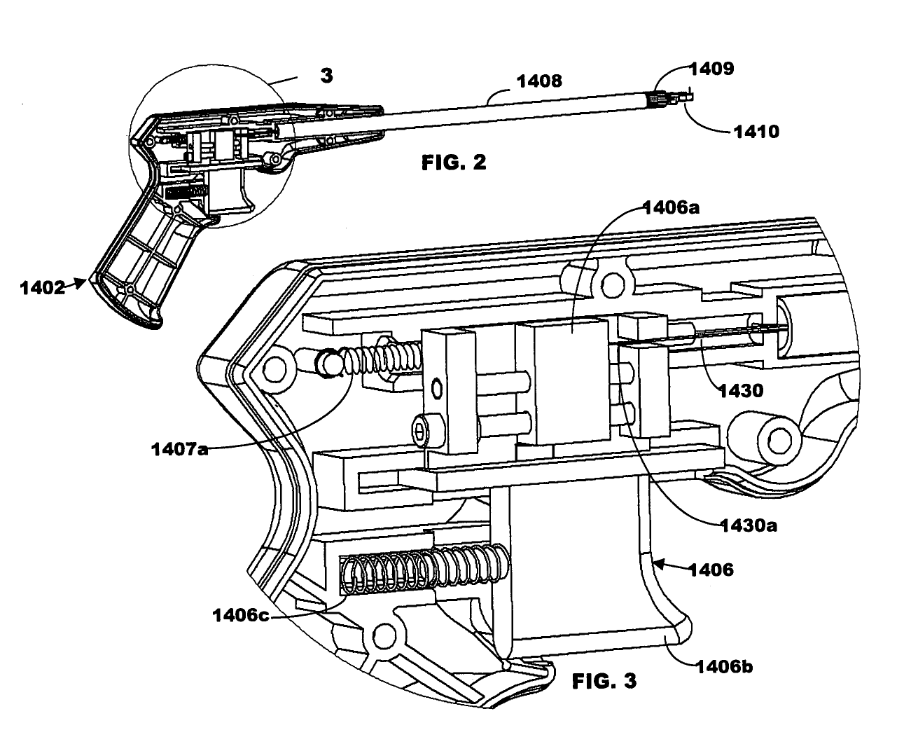

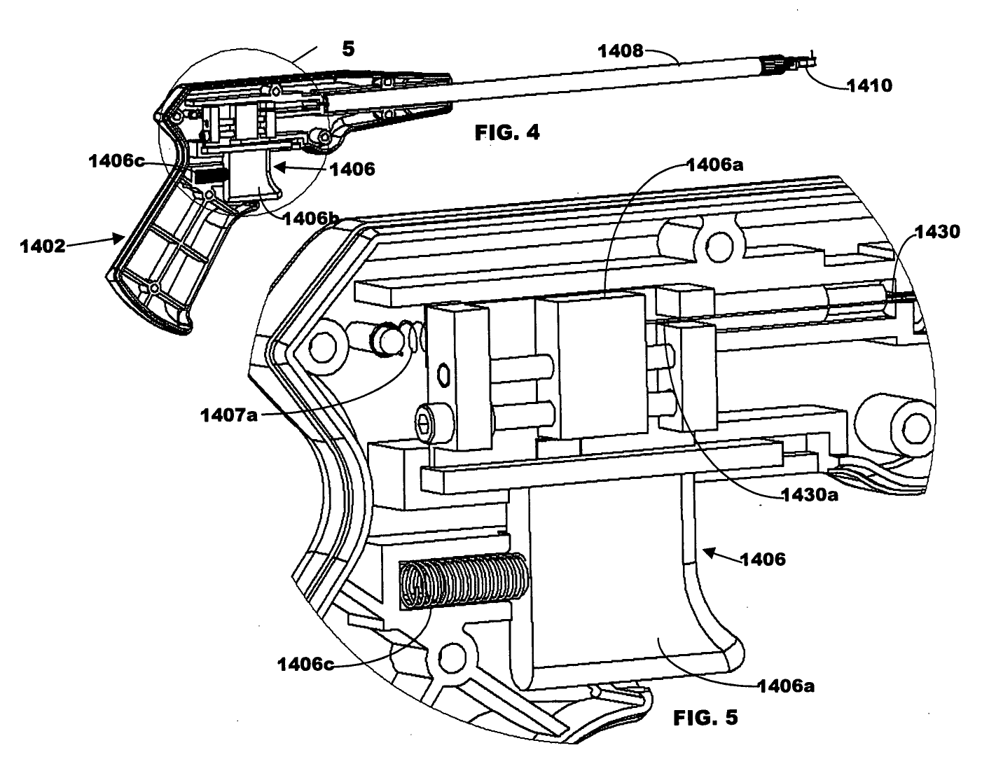

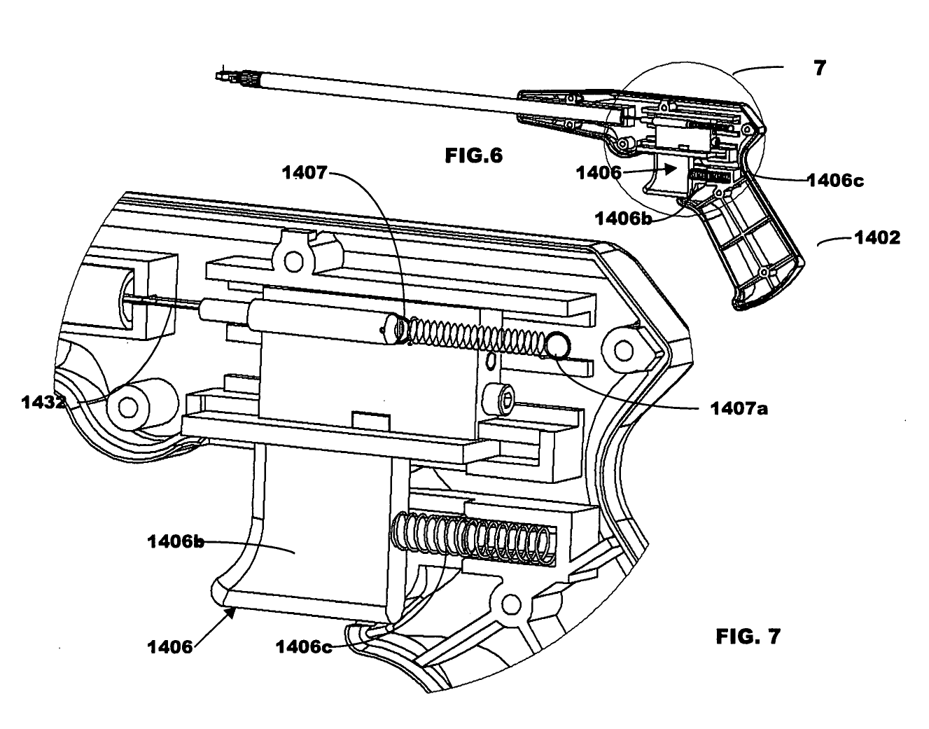

Turning to FIGS. 1 through 19 of the drawings, one form of the cycling suturing and knot-tying device of this invention is there illustrated and generally identified by the numeral 1400. Referring particularly to FIG. 1 of the drawings, device 1400 can be seen to comprise a gripping portion 1402 comprising a generally pistol-shaped handgrip 1404 and a trigger mechanism 1406 connected to the handgrip in the manner shown in FIGS. 2, 4 and 6. Trigger mechanism 1406 comprises a part of the novel operating means of the invention, the character of which will presently be described.

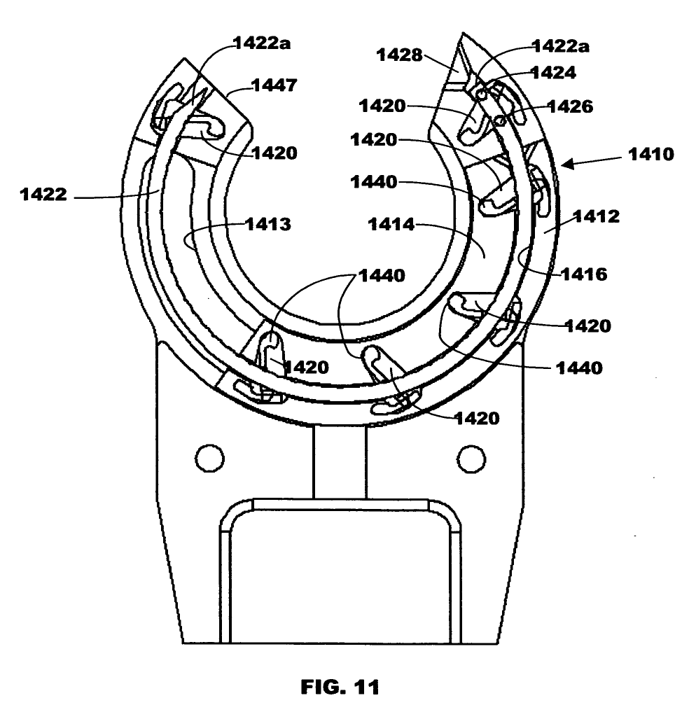

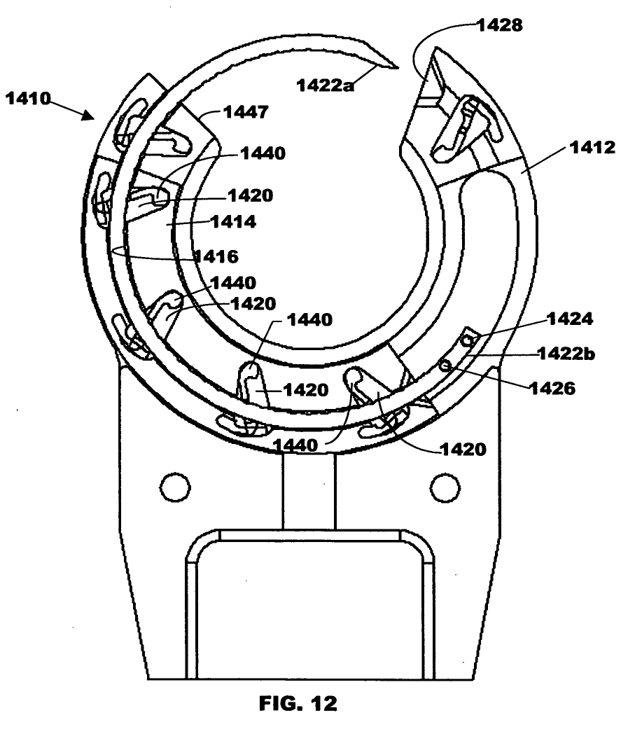

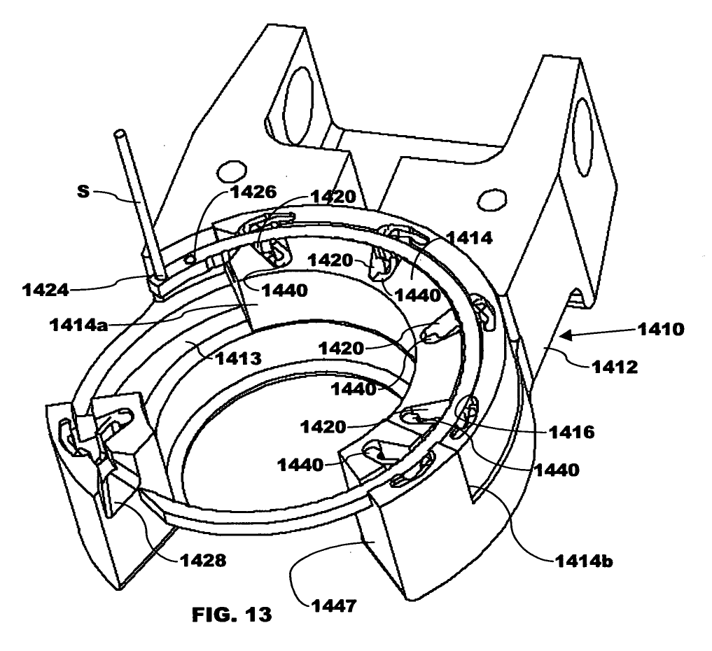

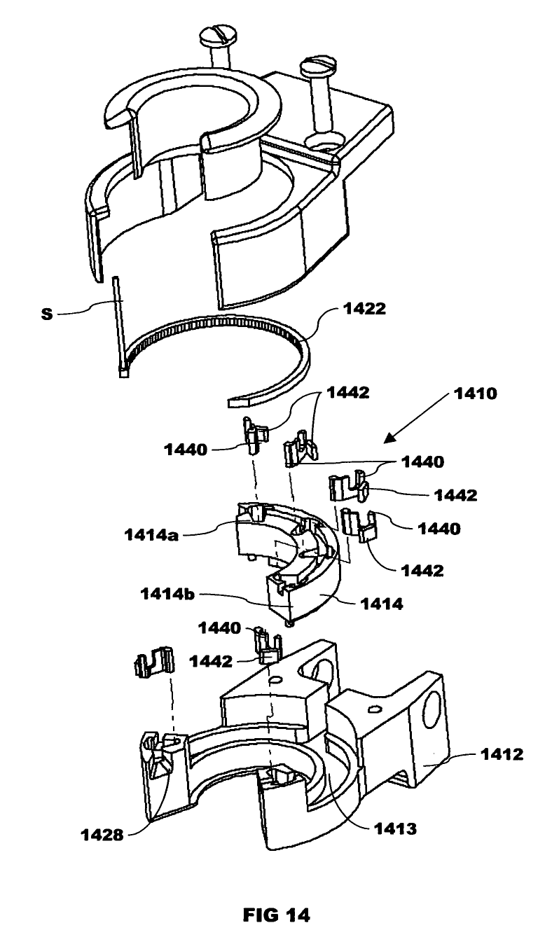

Connected to gripping portion 1402 is an elongated, hollow-barrel portion 1408 and connected to the hollow-barrel portion is an articulating, suturing head portion generally designated by the numeral 1410. Articulating-head portion 1410, which comprises one of the improved features of this latest form of the invention, is of a novel design that includes a generally semi-circular-shaped body 1412 having a generally semi-circular-shaped shuttle track 1413 (FIGS. 11 and 12). Operably associated with body 1412 is a generally semi-circular-shaped shuttle member 1414 that is slidably movable by the operating means of the invention along the shuttle track between a first position shown in FIG. 11 and the advanced second position shown in FIG. 12. As best seen in FIGS. 13 and 14, shuttle member 1414, which has a first end 1414a and a second end 1414b, is provided with a generally semi-circular-shaped needle groove or guide 1416 that extends from the first end of the shuttle member to the second end thereof. Uniquely, shuttle member 1414 is also provided with a plurality of strategically shaped, circumferentially spaced cavities 1420 the purpose of which will be described in the paragraphs which follow.

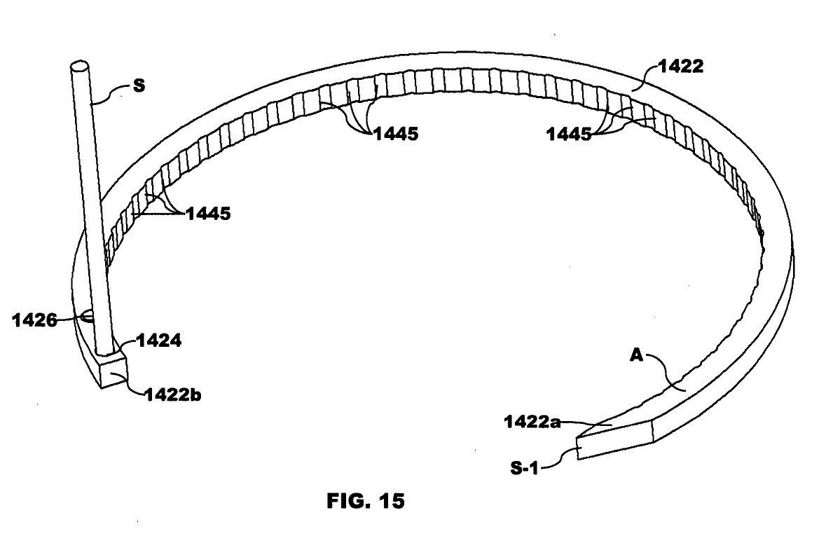

Carried within a needle guide 1416 that is formed in shuttle member 1414 is a highly novel, generally semi-circular-shaped suturing needle 1422. Needle 1422, which can be constructed from metal or plastic, is incrementally movable along the needle guide from a first position shown in FIG. 11 to an advanced second position shown in FIG. 12 and then to a third further advanced position. As best seen in FIG. 15, needle 1422, which has first and second ends 1422a and 1422b, is of a unique construction. Unlike most prior art suture needles, needle 1422, rather than being circular in cross section, is generally rectangular in cross section and has upper and lower surfaces disposed within the generally parallel planes (See FIG. 15). The first end of the suture needle is chamfered at a precisely selected angle, while the second end thereof is provided with a pair of spaced-apart apertures 1424 and 1426. These apertures, which receive the suture “S” extend generally perpendicular to the plane of the upper and lower surfaces of the needle. To counter the tendency of the needle to open up as it penetrates the tissue to be sutured, the point “S−1” of the needle is off-center of the axis “A” of the arc of the needle (see FIG. 15).

As best seen in FIGS. 12 and 13, the first end 1412a of generally semi-circular-shaped body 1412 is provided with a generally conically shaped opening 1428 for receiving the chamfered end of the needle as the needle is incrementally advanced. The conically shaped opening 1428 is strategically configured so as to permit the chamfered end of the needle to deflect somewhat as it is guided into the groove or guide 1416 formed in the shuttle member.

Considering now in greater detail the previously mentioned operating means of this latest form of the invention, As will be understood from the discussion that follows, this novel operating means functions to controllably advance and retract the shuttle member 1414 along shuttle track 1413 between its first and second positions. This sequential movement of the shuttle member, in turn, uniquely causes the suturing needle 1422 to incrementally move smoothly along the needle guide from its first position to its second position and then onto further advanced positions within the shuttle head. In addition to the previously mentioned trigger mechanism 1406, this important operating means also comprises first and second operating cables 1430 end 1432 which are strategically entrained through hollow-barrel portion 1408 in the manner illustrated in FIGS. 2, 6 and 8. As seen in the drawings, operating cable 1430 has a first end 1430a connected proximate the first end 1414a of shuttle 1414 and a second end 1430b connected to trigger mechanism 1406. Similarly, second operating cable 1432 has a first end 1432a connected proximate second end 1414b of shuttle 1414 and a second end 1432b connected to trigger mechanism 1406.

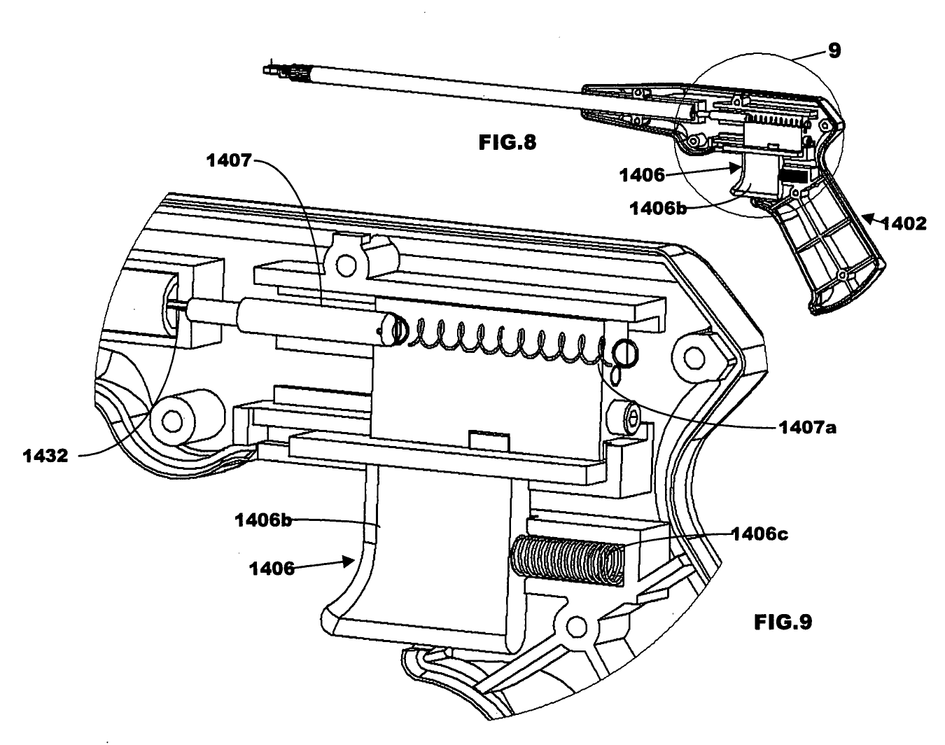

With the construction described in the preceding paragraph, sequential actuation and release of the trigger of the trigger mechanism will cause the shuttle to sequentially move along the shuttle track between the first and second positions in the manner illustrated in FIGS. 11 and 12. More particularly, when the trigger of the trigger mechanism is actuated, the first operating cable 1430 will move the shuttle 1414 in a clockwise direction from the first position shown in FIG. 11 to the second position shown in FIG. 12. As this occurs, a biasing means, shown here as a compression spring 1406c, which is connected to a reciprocally movable coupling mechanism that is carried by gripping portion 1402, yieldably resists movement of the trigger, is extended as illustrated in FIG. 8. In its extended position the extension spring acts upon the second operating cable 1432 via the coupling mechanism in the manner such that when the trigger is released, the shuttle 1414 will be caused to move in a counterclockwise direction and return to its starting position.

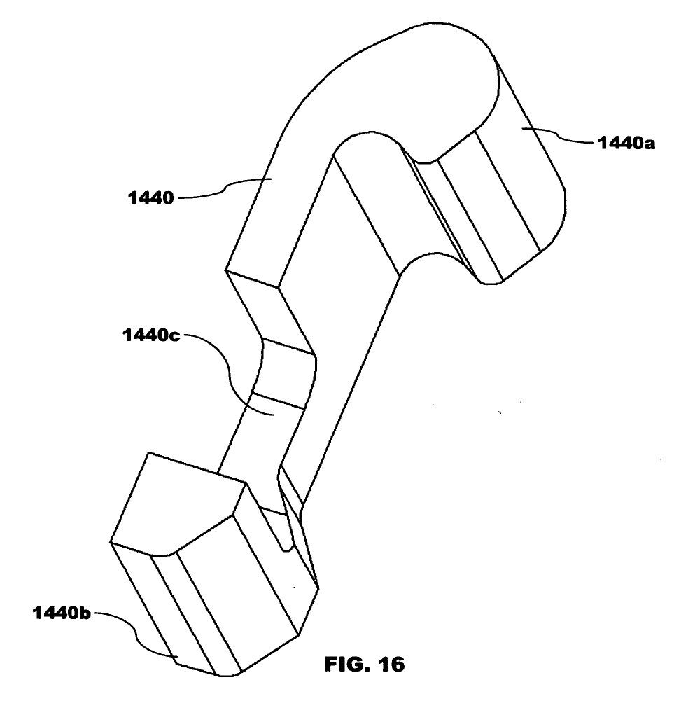

In a manner now to be described, movement of the shuttle 1414 along the shuttle track 1413 causes concomitant, controlled movement of the suture needle 1422 along needle guide 1416. As previously mentioned, shuttle member 1414 is provided with a plurality of strategically shaped, circumferentially spaced cavities 1420. Disposed within each of these cavities 1420 is a uniquely configured needle-engaging member 1440 (see FIG. 16) that is adapted for both transverse and pivotal movement within the cavity in response to movement of the 1414 shuttle between its first and second positions. This novel movement of the members 1440 within the cavities 1420 is illustrated in FIGS. 17 and 18 of the drawings. As shown in FIG. 17, also partially disposed within cavities 1420 are biasing means, shown here as compressible, expandable elastomeric springs 1442, which act upon members 1440. Springs 1442, which are of a generally cylindrically shaped, plug-like configuration are carried within smaller cavities segments 1444 which communicate with larger cavities 1420 in the manner illustrated in FIGS. 17 and 18.

Turning particularly to FIG. 15, it is to be noted that suturing needle 1422 is provided with a multiplicity of circumferentially spaced-apart notches 1445, which are uniquely constructed and arranged to be engaged by the needle-engaging members as the needle-engaging members move within cavities 1420. More particularly, as the shuttle member 1414 moves from the first position shown in FIG. 11 toward the second position shown in FIG. 12, the needle-engaging drive members will engage the needle in the manner illustrated in FIG. 17, causing the needle 1422 to move along with the shuttle member and penetrate the tissue disposed within the head opening 1447 (FIG. 10). Unlike the prior art circular cross section suturing needles, which provide only a point contact with a needle driving member, the novel rectangular cross section needle of the present invention presents a substantially flat, grooved wall that provides a superior line contact with the driving member that advances the needle.

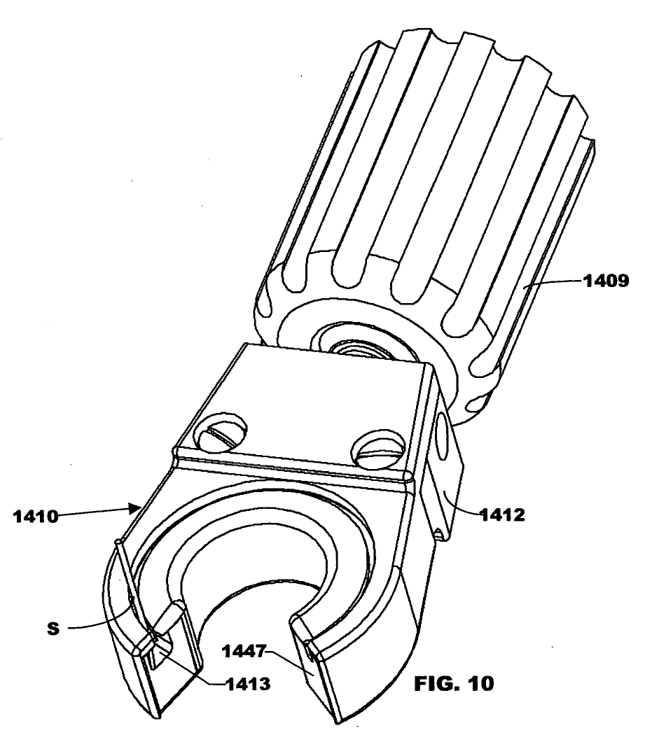

As indicated in FIG. 17, spring 1442 continuously urges the drive members into binding engagement with the needle. However, upon release of the trigger, which permits the shuttle to return to its starting position due to the urging of the extension spring 1407a (FIG. 9), the needle-engaging members 1440 will compress the elastomeric springs 1442 and will pivot and move transversely within cavities 1420 in the direction of the arrows to engage the needle in the manner shown in FIG. 18 to allow the members 1440 to slide relative to the needle allowing the needle to remain in place when the trigger is again actuated, the shuttle member 1414 will once again move in a clockwise direction as illustrated in FIG. 12 causing the needle-engaging members 1440 to once again grip the suturing needle 1422 due to the urging of the elastomeric springs 1442. This gripping of the needle will once again cause it to advance in a clockwise direction along the needle guide 1416 toward its third advanced position (not shown). As the process is repeated, the needle will continue to advance in a clockwise direction along the needle guide 1416 so that the suturing can be controllably and efficiently completed.

As illustrated in FIGS. 11, 12 and 13, body 1412 of the suturing head is also provided with a pair of strategically shaped, circumferentially spaced cavities 1420 within which needle-engaging members 1440 are housed. These members cooperate with and function in an identical manner as the needle-engaging members housed within the cavities formed in the shuttle 1414 to control the movement of the suturing needle within guide-way 1416 as the shuttle moves along the shuttle track 1413. More particularly, as the shuttle member 1414 moves from the first position shown in FIG. 11 toward the second position shown in FIG. 12, these needle-engaging members will engage the needle in the manner illustrated in FIG. 17, allowing the needle 1422 to move with the shuttle member. However, upon release of the trigger, which permits the shuttle to return to its starting position due to the urging of the extension spring 1407c, these needle-engaging drive members will move into the needle slip configuration shown in FIG. 18 permitting the needle to remain in its advanced position.

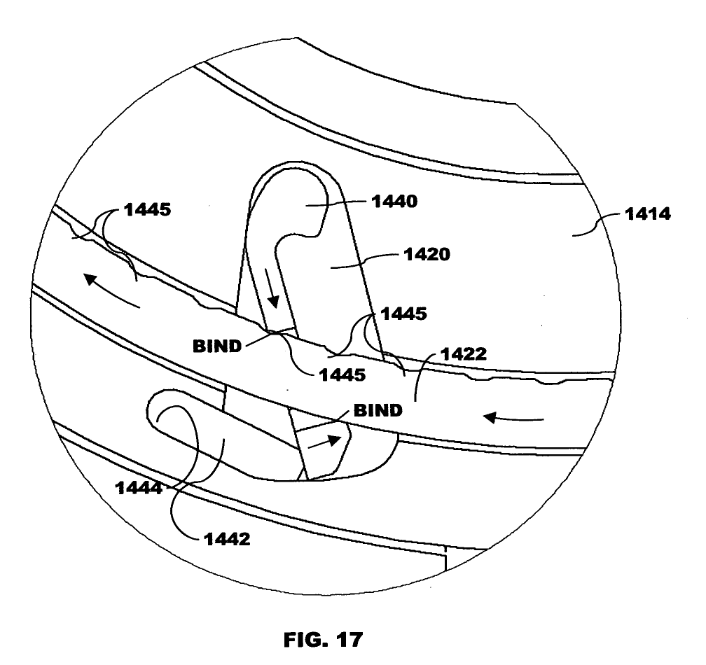

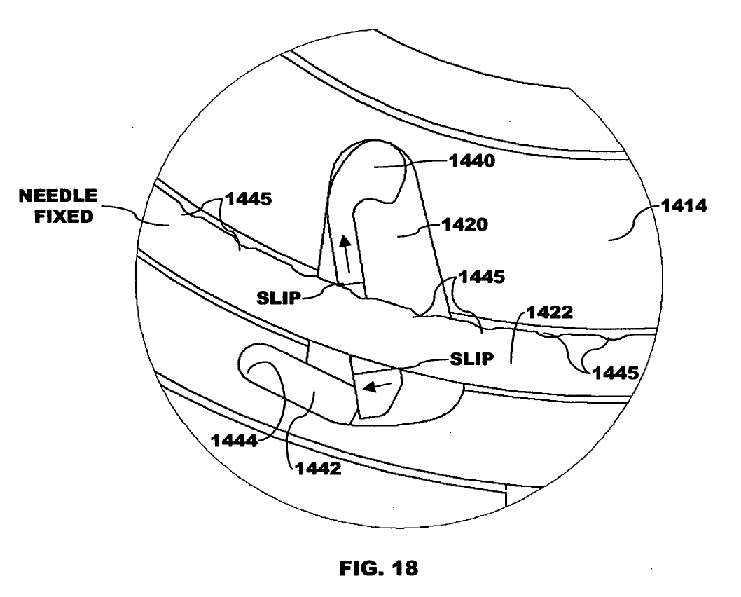

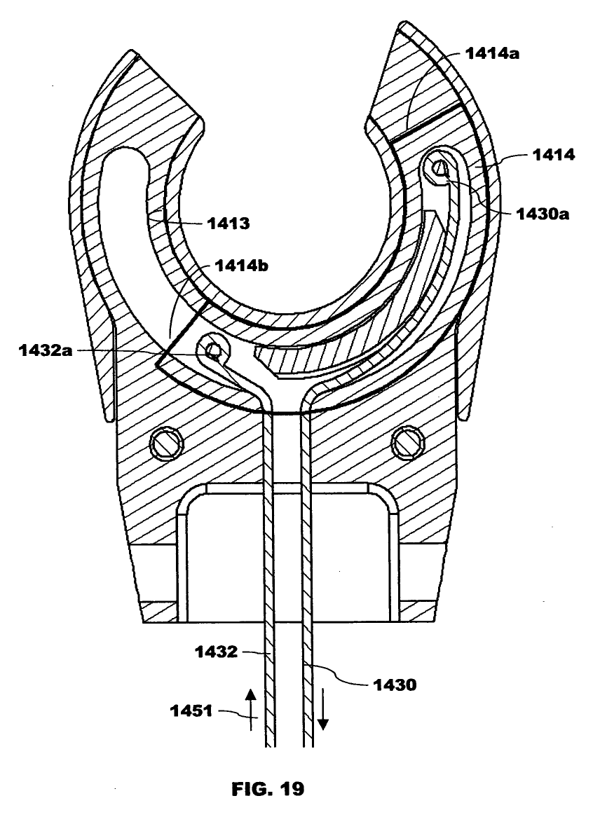

In using the suturing device of the present invention, with the suturing head components in the position illustrated in FIG. 11 and with the tissue to be sutured disposed within open 1447, the suturing process is begun by actuating the trigger of the trigger mechanism. When the trigger is actuated, the first operating cable 1430, which is connected proximate the bottom of the first end of the shuttle 1414 (see FIG. 19), will move the shuttle 1414 in a clockwise direction from the first position shown in FIG. 11 to the second position shown in FIG. 12. As the shuttle moves in this clockwise direction, cable 1432 will be foreshortened in the direction of the arrow 1451 of FIG. 19 causing the extension spring 1407c to be extended by the reciprocally movable coupling mechanism in the manner shown in FIG. 8.

During the clockwise movement of the shuttle, elastomeric springs 1442 will urge spring engaging members 1440 into binding engagement with the needle 1422 in the manner illustrated in FIG. 17 causing the needle, along with the suture “S”, to advance to the needle penetrating position shown in FIG. 12. When the needle and the shuttle reaches this advanced position, the trigger is released thereby permitting the shuttle 1414 to move in a counterclockwise direction toward its starting position due to the urging of extension spring 1407c. During this counterclockwise movement of the shuttle members, the needle-engaging members 1440 will move within cavities 1420 into the needle slip position illustrated in FIG. 18. This novel pivotal and transverse movement of the needle-engaging members within their respective cavities will compress elastomeric springs 1442 and will permit the needle 1422 to slip relative to the shuttle members and remain in the advanced position shown in FIG. 12.

After the shuttle members return to their starting positions, actuation of the trigger member will once again cause clockwise movement of the shuttles along the shuttle track 1413. As before, during this clockwise movement of the shuttle, elastomeric springs 1442 will urge spring engaging members 1440 into binding engagement with the needle 1422 in the manner illustrated in FIG. 17 causing the needle and the suture “S” to advance to a third, further advanced position (not shown). It is to be appreciated that by the repeated actuation and release of the trigger member the suturing needle can be smoothly and controllably, incrementally advanced along the needle guide 1416 to efficiently complete the suturing operation.

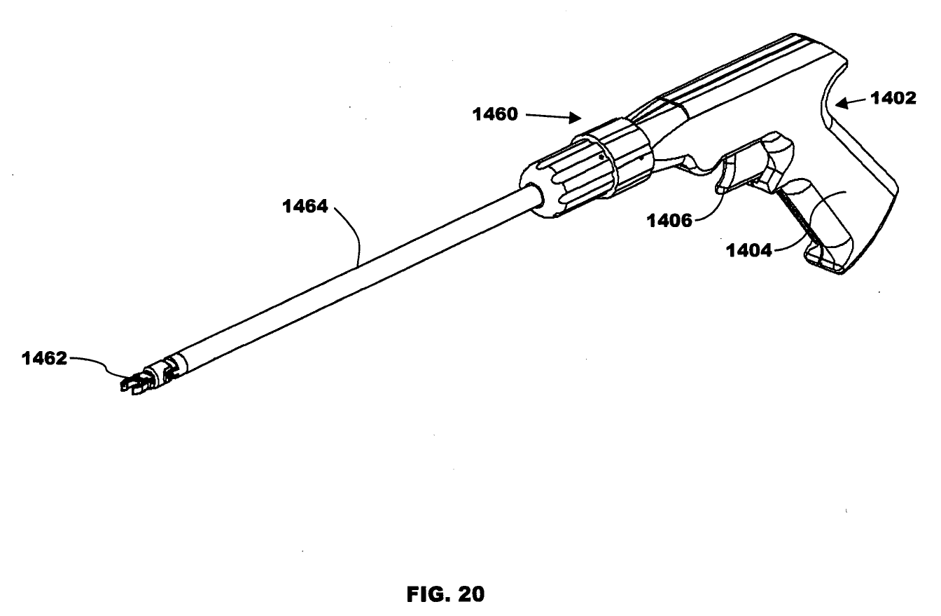

Turning to FIGS. 20 through 32 of the drawings, an alternate form of the cycling, suturing and knot-tying device of this invention is there illustrated and generally identified by the numeral 1460. This embodiment is similar in some respects to the embodiments described in U.S. Pat. No. 7,004,951, but includes several improvements the nature of which will be discussed in the paragraphs which will follow. Because of its pertinence, U.S. Pat. No. 7,004,951 is hereby incorporated by reference as though fully set forth herein. This latest embodiment of the invention is also similar in some respects to that shown in FIGS. 1 through 19 of the present application and like numerals are used in FIGS. 20 through 32 to identify like components. The primary differences between this embodiment and that of FIGS. 1 through 19 reside in the somewhat differently configured suturing head 1462 and the provision of novel suturing head-positioning means that allows the suturing head to be controllably moved arcuately relative to hollow-barrel portion 1464 to which it is connected.

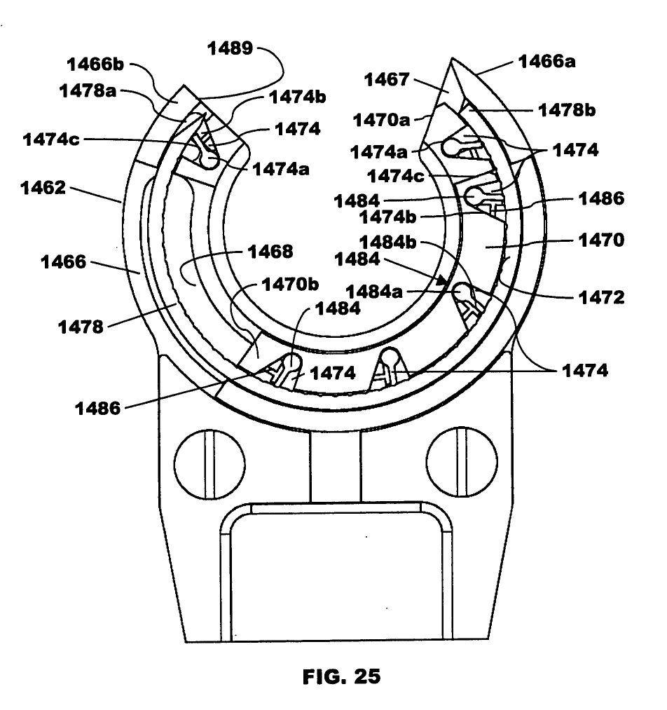

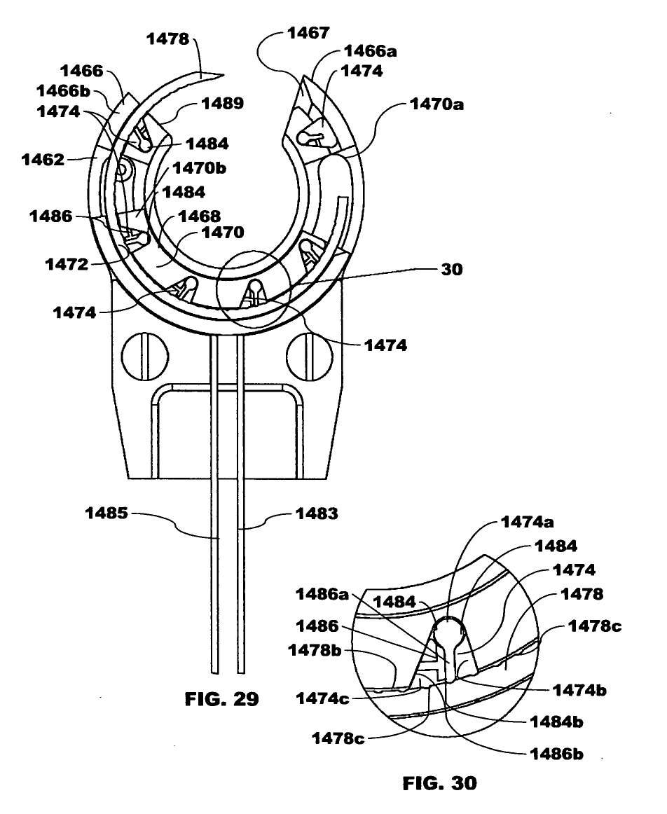

Referring particularly to FIG. 20 of the drawings, device 1460 can be seen to include a gripping portion 1402 that comprises a generally pistol-shaped handgrip 1404 and a trigger mechanism 1406 connected to the handgrip in the manner shown in FIGS. 20 and 21. Trigger mechanism 1406, which is substantially identical in construction and operation to that previously described, comprises a part of the novel operating means of this latest form of the invention. Connected to gripping portion 1402 is the elongated, barrel assembly 1464 and connected to the hollow-barrel assembly is an articulating, suturing head portion 1462. Articulating-head portion 1462, which comprises one of the improved features of this latest form of the invention, is of a novel design that includes a generally semi-circular-shaped body 1466 having a semi-circular-shaped shuttle track 1468 and first and second end portions 1466a and 1466b (FIG. 25). Operably associated with body 1466 is a generally semi-circular-shaped shuttle member 1470 that is slidably movable by the operating means of the invention along the shuttle track between a first position shown in FIG. 25 and a second position shown in FIG. 29. As illustrated in FIG. 25, shuttle member 1470, which has a first end 1470a and a second end 1470b, is provided with a generally semi-circular-shaped needle groove or guide 1472 that extends from the first end of the shuttle member to the second end thereof. Uniquely, shuttle member 1470 as well as end portions 1466a and 1466b of body 1466 are provided with strategically shaped, circumferentially spaced cavities 1474 the purpose of which will be described in the paragraphs which follow.

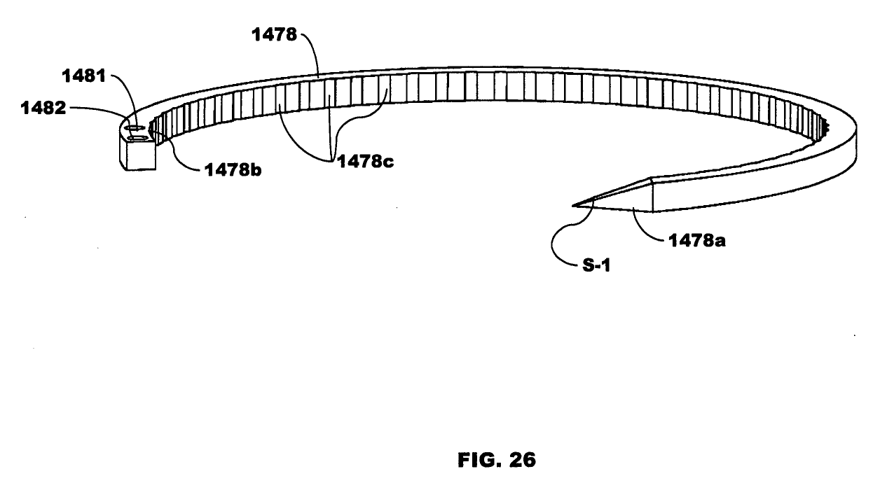

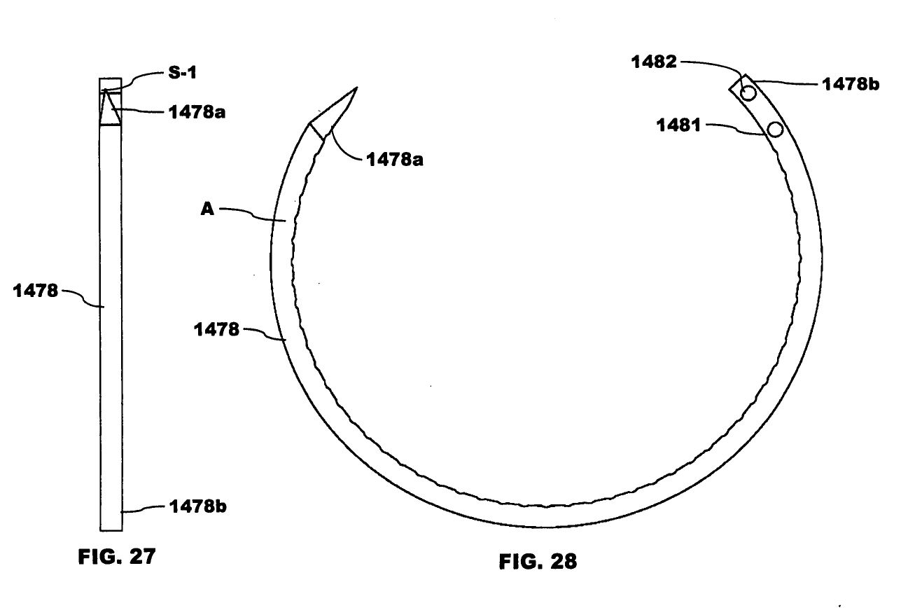

Carried within a needle guide 1472 is a generally semi-circular-shaped suturing needle 1478. Needle 1478, which is similar to the earlier described needle 1422, can be constructed from metal or plastic and is incrementally movable along the needle guide from a first position shown in FIG. 25 to a second position shown in FIG. 29 and then to a third, further advanced position. As best seen in FIGS. 26 and 28, needle 1478 has first and second ends 1478a and 1478b and, rather than being circular in cross section, is generally rectangular in cross section and has upper and lower surfaces disposed within generally parallel planes (See FIG. 27). The first end of the suture needle is chamfered at a precisely selected angle, while the second end thereof is provided with a pair of spaced-apart apertures 1481 and 1482. These apertures, which receive the suture “S” (FIG. 13) extend generally perpendicular to the plane of the upper and lower surfaces of the needle. To counter the tendency of the needle to open up as it penetrates the tissue to be sutured, the point “S−1” of the needle is off-center of the axis “A” of the arc of the needle (see FIG. 28).

As best seen in FIGS. 25 and 29, the first end 1466a of generally semi-circular-shaped body 1466 is provided with a generally conically shaped opening 1467 for receiving the chamfered end of the needle as the needle is incrementally advanced. The conically shaped opening 1466a is strategically configured so as to permit the chamfered end of the needle to deflect somewhat as it is guided into the groove or guide 1472 formed in the shuttle member.

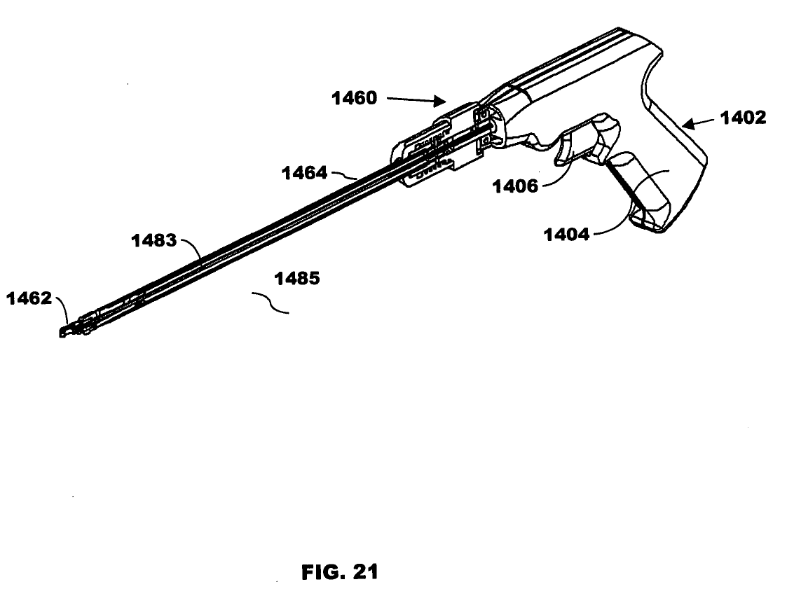

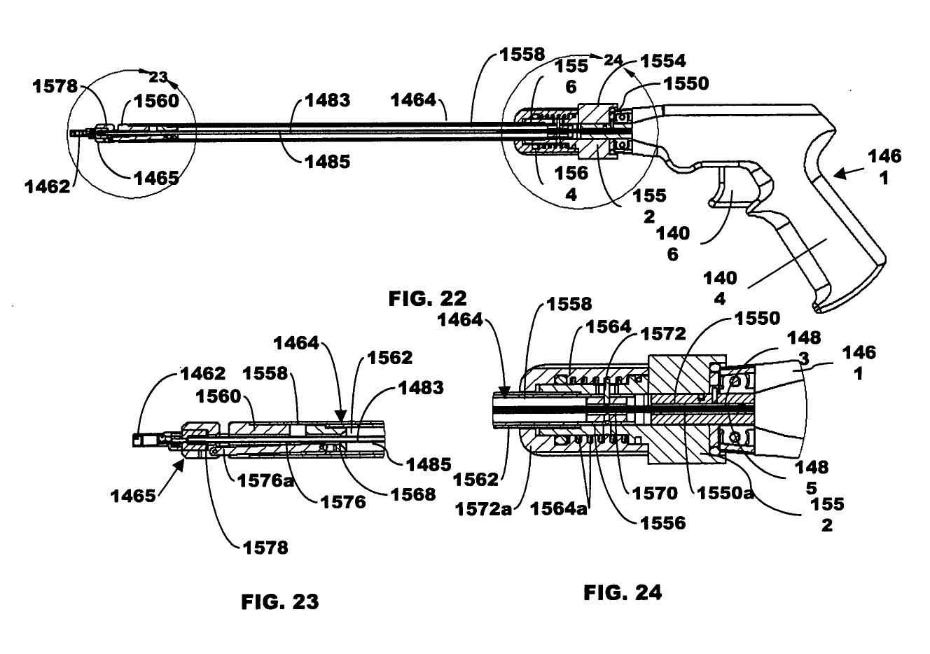

Considering now in greater detail the operating means of this latest form of the invention, as will be understood from the discussion that follows, this novel operating means functions to controllably advance and retract the shuttle member 1470 along shuttle track 1468 between its first and second positions. This sequential movement of the shuttle member, in turn, uniquely causes the suturing needle 1478 to incrementally move smoothly along the needle guide from its first position to its second position and then onto further advanced positions within the shuttle head. In addition to the previously mentioned trigger mechanism 1406, this important operating means also comprises first and second operating cables 1483 and 1485 which are strategically entrained through hollow-barrel portion 1408 in the manner illustrated in FIGS. 21 and 22. First and second operating cables 1483 and 1485 (FIG. 29) perform a similar function and are of similar construction to the previously described operating cables 1430 and 1432. More particularly, as in the last described embodiment, the operating cables include a first cable having a first end connected proximate the first end of the shuttle (see FIG. 19) and a second end connected to a coupling mechanism of trigger mechanism 1406 (see FIG. 3). Similarly, in this latest embodiment, the operating cables include a second operating cable having a first end connected proximate the second end of the shuttle (see FIG. 19) and a second end connected to a return mechanism 1407 which includes a biasing means or return spring 1407a that is connected to the gripping portion 1402 (See FIG. 7).

With the construction illustrated in the drawings and described in the preceding paragraph, sequential actuation and release of the trigger of the trigger mechanism will cause the shuttle to sequentially move along the shuttle track 1468 between the first and second positions in the manner illustrated in FIGS. 25 and 29. More particularly, when the trigger of the trigger mechanism is actuated, the first operating cable 1483 will move the shuttle 1470 in a clockwise direction from the first position shown in FIG. 25 to the second position shown in FIG. 29. As this occurs, the biasing means, or return spring 1407a of the return mechanism 1407, which is connected to the reciprocally movable coupling mechanism, is extended as illustrated in FIG. 9. In its extended position the extension spring acts upon the second operating cable 1483 tending to return it to its starting position and, in turn, tending to move the shuttle 1470 in a counterclockwise direction toward its starting position. To return the trigger to its starting position following trigger actuation a compressible coil spring 1406c is provided. Spring 1406c, which comprises a part of the trigger mechanism 1406, is compressed in the manner shown in FIG. 9 when the trigger is actuated and functions to return the trigger to its default or starting position shown in FIG. 9 when pressure on the trigger is released.

In a manner now to be described, movement of the shuttle 1470 along the shuttle track 1468 causes concomitant, controlled movement of the suture needle 1478 along needle guide 1472. As previously mentioned, shuttle member 1470 as well as end portions 1466a and 1466b are provided with a plurality of strategically shaped, circumferentially spaced cavities 1474, each of which includes a rounded apex 1474a and angularly extending side walls 1474b and 1474c (FIG. 30). Disposed within each of these cavities 1474 is a uniquely configured needle drive means for driving the suture needle 1478 along needle guide 1472. This needle drive means here comprises a needle-engaging member 1484 and a generally “T”-shaped, elastomeric return member 1486 that is operably associated with member 1484. As best seen in FIGS. 30 and 32, needle-engaging member 1484 includes a rounded-head portion 1484a that is pivotally received within the rounded apex 1474a of the cavity 1474 and an outwardly extending needle-engaging leg 1484b. As illustrated in FIG. 30, return member 1486, which comprises the return means of this form of the invention to continuously, yieldably urge the free end of needle-engaging leg 1484b into a first position in engagement with a selected one of a plurality of circumferentially spaced-apart notches 1478c formed on the inner surface 1478b of needle 1478 (see FIG. 26). More particularly, return member 1486 has an elongated portion 1486a that is maintained in engagement with member 1484 and a yieldably deformable leg portion 1486b that is normally maintained in engagement with wall 1474b of cavity 1474.

With the construction thus described, as the shuttle member 1470 moves from the first position shown in FIG. 25 toward the second position shown in FIG. 29, the needle-engaging members will engage the needle in the manner illustrated in FIGS. 25 and 30, causing the needle 1478 to move along with the shuttle member and to penetrate the tissue disposed within the head opening 1489. Unlike the prior art circular cross section suturing needles, which provide only a point contact with a needle driving member, the novel rectangular cross section needle of the present invention presents a substantially flat, grooved or notched wall that provides a superior line contact with the driving member that advances the needle.

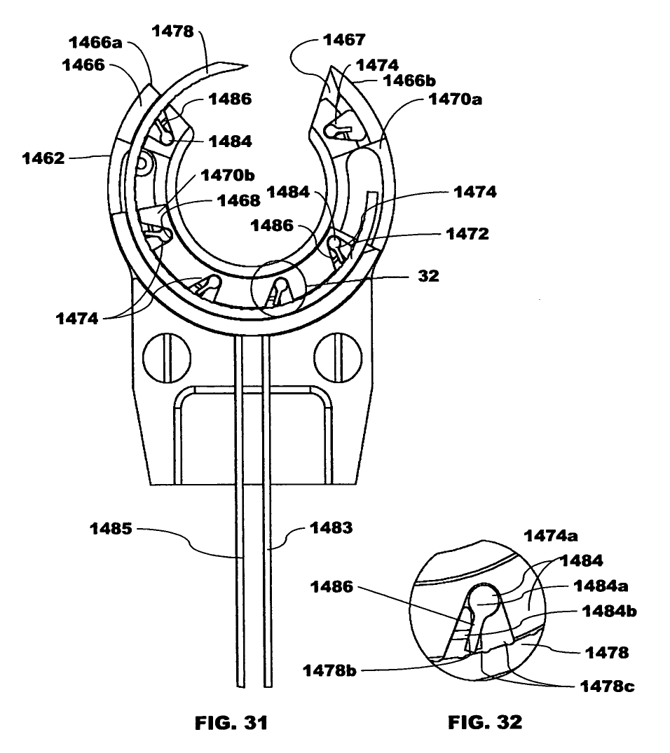

As indicated in FIG. 30, the return means, or member 1486 of the device continuously urges the needle-engaging members 1484 into binding engagement with the needle. However, upon release of the trigger, which causes the shuttle to return to its starting position due to the urging of the extension spring 1407, the return members that are disposed within the cavities of 1474 will yieldably deform in a manner to permit the needle-engaging members to pivot into the second position shown in FIGS. 31 and 32. With the needle-engaging members in this second position, as the trigger is released causing the shuttle member 1470 to move counterclockwise to the position illustrated in FIG. 25, the needle-engaging members will slide relative to the needle. However, the needle-engaging members disposed within the cavity 1474 formed in end portion 1466b will not deform and, accordingly, will hold the needle in place. When the trigger is once again actuated, the shuttle member 1470 will again move in a clockwise direction as illustrated in FIG. 29 causing the needle-engaging members to once again grip and further advance the suturing needle 1478. As the process is repeated, the needle will continue to advance in a clockwise direction along the needle guide 1472 so that the suturing can be controllably and efficiently completed.

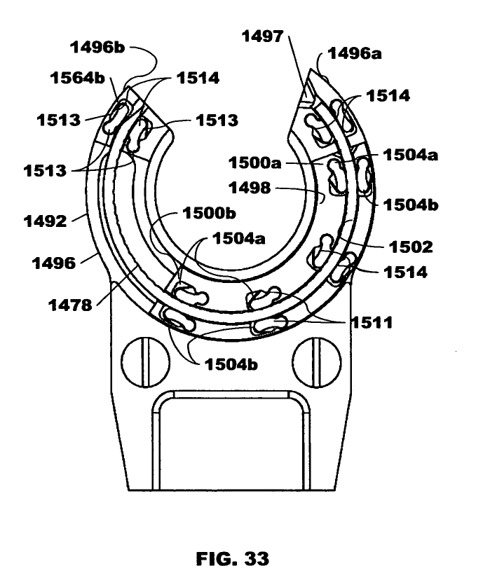

Turning to FIGS. 33 through 38 of the drawings, still another form of the cycling, suturing and knot-tying device of this invention is there illustrated. This embodiment is similar in many respects to the embodiment illustrated in FIGS. 20 through 32 and like numerals are used in FIGS. 33 through 38 to identify like components. The primary differences between this embodiment and that of FIGS. 1 through 19 resides in the somewhat differently configured articulating, suturing head 1492 and the provision of differently configured drive means for advancing the semi-circular-shaped suturing needle 1478 within the suturing head.

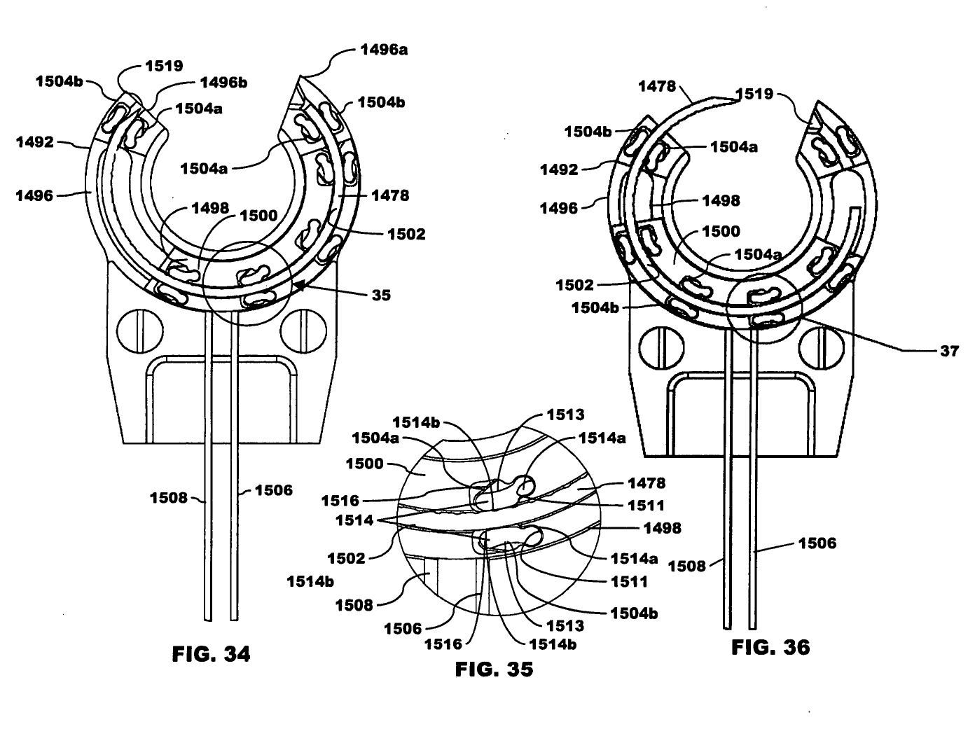

As best seen in FIG. 33, articulating-head portion 1492 here comprises a generally semi-circular-shaped body 1496 having first and second end portions 1496a and 1496b and a semi-circular-shaped shuttle track 1498. Operably associated with body 1496 is a generally semi-circular-shaped shuttle member 1500 that is slidably movable by the operating means of the invention along the shuttle track between a first position shown in FIGS. 33 and 34 and a second position shown in FIG. 36. As illustrated in FIG. 33, shuttle member 1500, which has a first end 1500a and a second end 1500b, is provided with a generally semi-circular-shaped needle grove or guide 1502 that extends from the first end of the shuttle member to the second end thereof. Uniquely, shuttle member 1500 is also provided with a plurality of strategically shaped, circumferentially spaced cavities 1504a and 1504b the purpose of which will be described in the paragraphs which follow.

Carried within needle guide 1502 is a generally semi-circular-shaped suturing needle 1478, which is substantially identical to the earlier described needle. Suturing needle 1478 is incrementally movable along the needle guide from a first position shown in FIG. 33 to a second position shown in FIG. 36 and then to a third, further advanced position.

As before, the first end 1496a of generally semi-circular-shaped body 1496 is provided with a generally conically shaped opening 1497 for receiving the chamfered end of the needle as the needle is incrementally advanced. The conically shaped opening 1497 is strategically configured so as to permit the chamfered end of the needle to deflect somewhat as it is guided into the groove or guide 1502 formed in the shuttle member.

The operating means of this latest form of the invention, which functions to controllably advance and retract the shuttle member 1500 along shuttle track 1498 between its first and second positions, is similar in most respects to that earlier described herein. In addition to the gripping portion 1402 and trigger mechanism 1406 carried by the handgrip, both of which are identical to those previously described, the operating means here comprises first and second operating cables 1506 end 1508 (FIG. 34) which are strategically entrained through hollow-barrel portion 1408 of the device in the same manner as illustrated in FIGS. 21 and 22 of the drawings. First and second operating cables 1506 and 1508 perform a similar function and are of similar construction to the previously described operating cables 1430 and 1432. More particularly, as in the last described embodiment, the operating cables include a first cable having a first end connected proximate the first end of the shuttle (see FIG. 19) and a second end connected to a coupling mechanism or trigger mechanism 1406 (see FIG. 3). Similarly, in this latest embodiment, the operating cables include a second operating cable having a first end connected proximate the second end of the shuttle (see FIG. 19) and a second end connected to a return mechanism 1407 which includes a biasing means or return spring 1407a that is connected to the gripping portion 1402 (See FIG. 7). Connected to gripping portion 1402 is the elongated, hollow-barrel portion 1464 and connected to the hollow-barrel portion is the articulating, suturing head portion 1492.

As in the last described embodiment, sequential actuation and release of the trigger of the trigger mechanism will cause the shuttle to sequentially move along the shuttle track 1498 between the first and second positions in the manner illustrated in FIGS. 33 and 36. More particularly, when the trigger of the trigger mechanism is actuated, the first operating cable 1506 will move the shuttle 1500 in a clockwise direction from the first position shown in FIG. 33 to the second position shown in FIG. 36. As this occurs, the biasing means, or return spring 1407a of the return mechanism 1407, which is connected to the reciprocally movable coupling mechanism, is extended as illustrated in FIG. 9. In its extended position the extension spring acts upon the second operating cable 1508 tending to return it to its starting position and, in turn, tending to move the shuttle 1500 in a counterclockwise direction toward its starting position. To return the trigger to its starting position following trigger actuation a compressible coil spring 1406c is provided. Spring 1406c, which comprises a part of the trigger mechanism 1406, is compressed in the manner shown in FIG. 9 when the trigger is actuated and functions to return the trigger to its default or starting position shown in FIG. 9 when pressure on the trigger is released.

As before, movement of the shuttle 1500 along the shuttle track 1498 causes concomitant, controlled movement of the suture needle 1478 along needle guide 1502. Shuttle member 1500 as well as end portions 1496a and 1496b are provided with a plurality of strategically shaped, circumferentially spaced cavities 1504a and 1504b, each of which includes a rounded socket-like portion 1511 and an open body portion 1513 (see FIGS. 34 and 35). Cavities 1504a and 1504b, which are positioned on opposite sides of needle passageway 1502, house uniquely configured needle drive means for driving the suture needle 1478 along needle guide 1502. This needle drive means here comprises a needle-engaging member 1514 that includes a rounded-head portion 1514a that is pivotally received within the rounded socket-like portions 1511 of the cavities and a body portion 1514b. The needle drive means also comprises return means shown here as a yieldably deformable spring-like return member 1516 that is operably associated with member 1514. As illustrated in FIG. 35, return member 1516 is constructed and arranged to continuously, yieldably urge the body portion 1514b of the driving member 1514 into driving engagement with needle 1478.

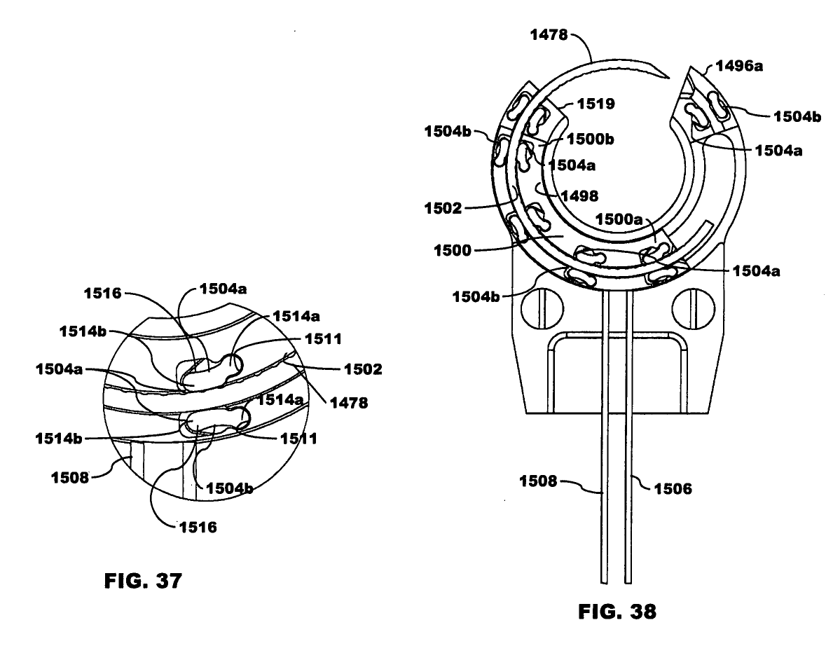

With the construction thus described, as the shuttle member 1500 moves from the first position shown in FIG. 34 toward the second position shown in FIG. 36, the needle-engaging members 1514 will engage both sides of the needle in the manner illustrated in FIG. 35 causing the needle 1478 to move along with the shuttle member and penetrate the tissue disposed within the head opening 1519. As indicated in FIG. 35, return members 1516 continuously urge the needle-engaging members 1514 into binding engagement with the needle. However, upon release of the trigger, which causes the shuttle to return to its starting position due to the urging of the extension spring 1407c, the return members that are disposed within cavities 1504a and 1504b formed in shuttle 1500 will yieldably deform in a manner to permit the needle-engaging members to pivot from their first driving position into their second position shown in FIGS. 36 and 37. With the needle-engaging members in this second position, as the trigger is released causing the shuttle member 1500 to move counterclockwise to the position illustrated in FIG. 34, the needle-engaging members will slide relative to the needle. However, the return members that are housed within the cavities 1504a and 1504b formed in the end portion 1504b will function to hold the needle in place in its advanced position. When the trigger is once again actuated, the shuttle member 1500 will again move in a clockwise direction as illustrated in FIG. 35 and the needle-engaging members will further advance the suturing needle 1478 into the position shown in FIG. 38. As the process is repeated, the needle will continue to advance in a clockwise direction along the needle guide 1502 so that the suturing can be controllably and efficiently completed.

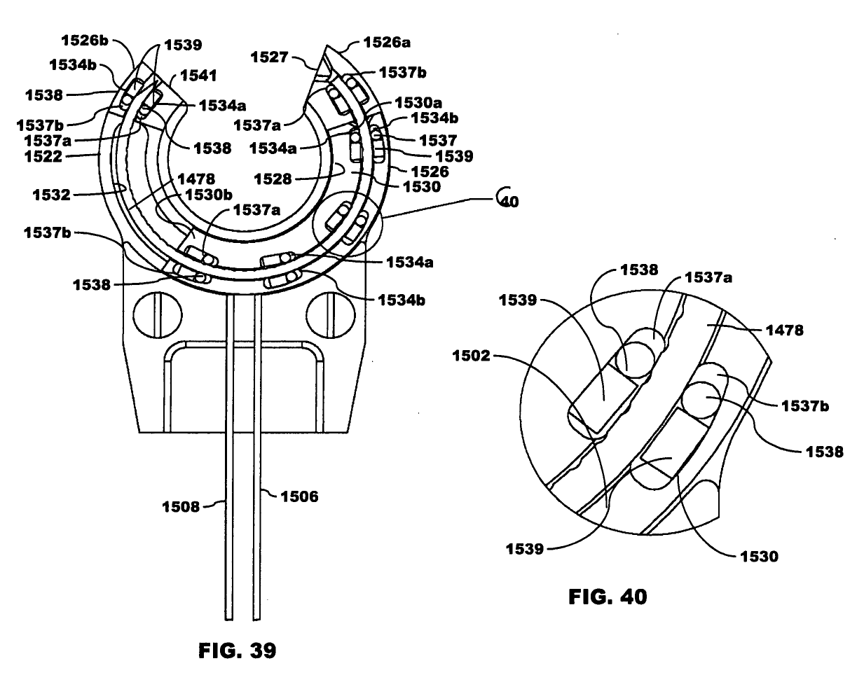

Turning to FIGS. 39 through 42 of the drawings, yet another form of the cycling, suturing and knot-tying device of this invention is there illustrated. This embodiment is similar in many respects to the embodiment illustrated in FIGS. 33 through 38 and like numerals are used in FIGS. 39 through 42 to identify like components. The primary differences between this embodiment and that of FIGS. 33 through 38 resides in the somewhat differently configured articulating, suturing head 1522 and the provision of differently configured drive means for advancing the semi-circular-shaped suturing needle 1478 within the suturing head.

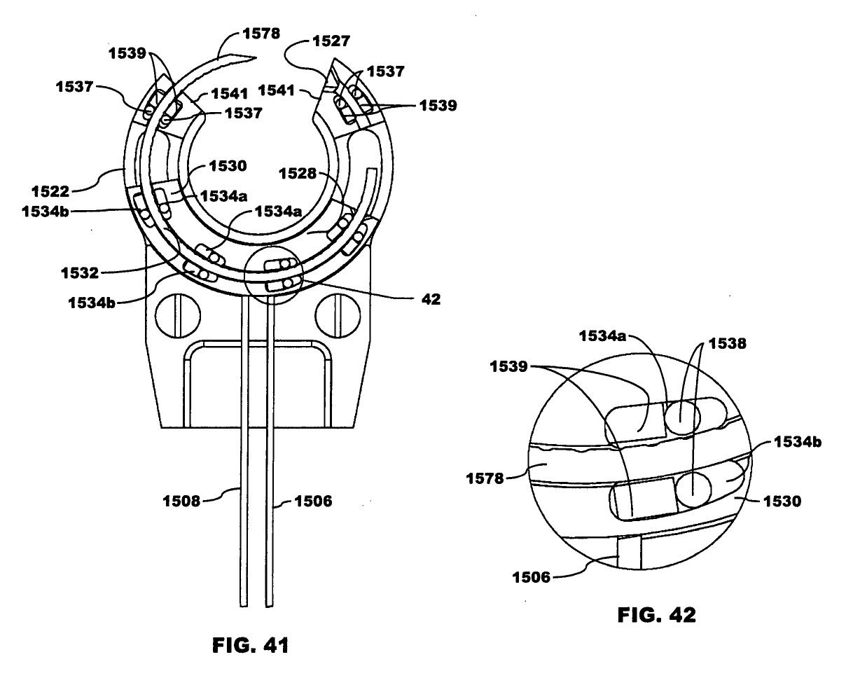

As best seen in FIG. 39, articulating-head portion 1522, here comprises a generally semi-circular-shaped body 1526 having end portions 1526a and 1526b and a semi-circular-shaped shuttle track 1528. Operably associated with body 1526 is a generally semi-circular-shaped shuttle member 1530 that is slidably movable by the operating means of the invention along the shuttle track between the position shown in FIG. 39 and the position shown in FIG. 41. As illustrated in FIG. 39, shuttle member 1530, which has a first end 1530a and a second end 1530b, is provided with a generally semi-circular-shaped needle groove or guide 1532 that extends from the first end of the shuttle member to the second end thereof. Uniquely, shuttle member 1530 is also provided with a plurality of strategically shaped, circumferentially spaced cavities 1534a and 1534b the purpose of which will be described in the paragraphs which follow.

Carried within needle guide 1532 is a generally semi-circular-shaped suturing needle 1478, which is substantially identical to the earlier described needle. Suturing needle 1478 is incrementally movable along the needle guide from a first position shown in FIG. 39 to a second position shown in FIG. 41 and then to a third, further advanced position.

As before, the first end 1526a of generally semi-circular-shaped body 1526 is provided with a generally conically shaped opening 1527 for receiving the chamfered end of the needle as the needle is incrementally advanced. The conically shaped opening 1527 is strategically configured so as to permit the chamfered end of the needle to deflect somewhat as it is guided into the groove or guide 1532 formed in the shuttle member.

The operating means of this latest form of the invention, which functions to controllably advance and retract the shuttle member 1530 along shuttle track 1528 between its first and second positions, is similar in most respects to that earlier described herein. In addition to the gripping portion 1402 and trigger mechanism 1406 carried by the handgrip, both of which are identical to those previously described, the operating means here comprises first and second operating cables 1506 end 1508 (FIG. 39) which are strategically entrained through hollow-barrel portion 1408 of the device in the same manner as illustrated in FIGS. 21 and 22 of the drawings. First and second operating cables 1506 and 1508 perform a similar function and are of similar construction to the previously described operating cables 1430 and 1432. More particularly, as in the last described embodiment, the operating cables include a first cable having a first end connected proximate the first end of the shuttle (see FIG. 19) and a second end connected to a coupling mechanism or trigger mechanism 1406 (see FIG. 3). Similarly, in this latest embodiment, the operating cables include a second operating cable having a first end connected proximate the second end of the shuttle (see FIG. 19) and a second end connected to a return mechanism 1407 which includes a biasing means or return spring 1407a that is connected to the gripping portion 1402 (See FIG. 7). Connected to gripping portion 1402 is the elongated, hollow-barrel portion 1464 and connected to the hollow-barrel portion is the articulating, suturing head portion 1462 (see FIG. 47).

As in the last described embodiment, sequential actuation and release of the trigger of the trigger mechanism will cause the shuttle to sequentially move along the shuttle track 1528 between the first and second positions in the manner illustrated in FIGS. 39 and 41. More particularly, when the trigger of the trigger mechanism is actuated, the first operating cable 1506 will move the shuttle 1530 in a clockwise direction from the position shown in FIG. 39 to the position shown in FIG. 41. As this occurs, the biasing means, or return spring 1407a of the return mechanism 1407, which is connected to the reciprocally movable coupling mechanism, is extended as illustrated in FIG. 9. In its extended position the extension spring acts upon the second operating cable 1508 tending to return it to its starting position and, in turn, tending to move the shuttle 1530 in a counterclockwise direction toward its starting position. To return the trigger to its starting position following trigger actuation a compressible coil spring 1406c is provided. Spring 1406c, which comprises a part of the trigger mechanism 1406, is compressed in the manner shown in FIG. 9 when the trigger is actuated and functions to return the trigger to its default or starting position shown in FIG. 9, when pressure on the trigger is released.

As before, movement of the shuttle 1530 along the shuttle track 1528 causes concomitant, controlled movement of the suture needle 1478 along needle guide 1532. Shuttle member 1530 as well as end portions 1526a and 1526b are provided with strategically shaped, circumferentially spaced, generally oval-shaped, tapered wall cavities 1537a and 1537b, each having opposing tapered side walls. Cavities 1537a and 1537b, which are positioned on opposite sides of needle passageway 1532, house uniquely configured needle drive means for driving the suture needle 1478 along needle guide 1532 (see FIGS. 40 and 42). This needle drive means here comprises generally cylindrically shaped, roller-like needle-engaging members 1538 that are closely held within the cavities for movement between a first needle-engaging, drive position shown in FIG. 40 and a second slip position shown in FIG. 42. As before, the needle drive means also comprises return means shown here as yieldably deformable return members 1539 that are operably associated with members 1538 and function to urge the driving members into their first position in engagement with needle 1478. More particularly, return members 1539 here comprise small lengths of silicone tubes that are constructed and arranged to continuously, yieldably urge the driving members 1538 to roll along the cavities in a manner to be cammed into driving engagement with needle 1478 (see FIG. 40).

With the construction thus described, as the shuttle member 1530 moves from the starting position shown in FIG. 39, the needle-engaging members 1538, which are being urged into cammed engagement with the needle in the manner illustrated in FIG. 40, will cause the needle 1478 to move along with the shuttle member and penetrate the tissue disposed within the head opening 1541. However, upon release of the trigger, which causes the shuttle to return to its starting position due to the urging of the extension spring 1407c, the return members that are disposed within the cavities 1537a and 1537b formed in shuttle member 1530 will yieldably deform in the manner shown in FIG. 40 to permit the needle-engaging members to roll into their second position shown in FIG. 40. With the needle-engaging members in this second position, as the trigger is released causing the shuttle member 1530 to move counterclockwise toward the starting position, the needle-engaging members will slide relative to the needle permitting the needle to remain in place. However, the needle-engaging members that are disposed within the cavities formed in end portion 1526b will function to hold the needle in place in its advanced position. When the trigger is once again actuated, the shuttle member 1530 will again move in a clockwise direction causing the needle-engaging members to once again grip and further advance the suturing needle. As the process is repeated, the needle will continue to advance in a clockwise direction along the needle guide 1532 so that the suturing can be controllably and efficiently completed.

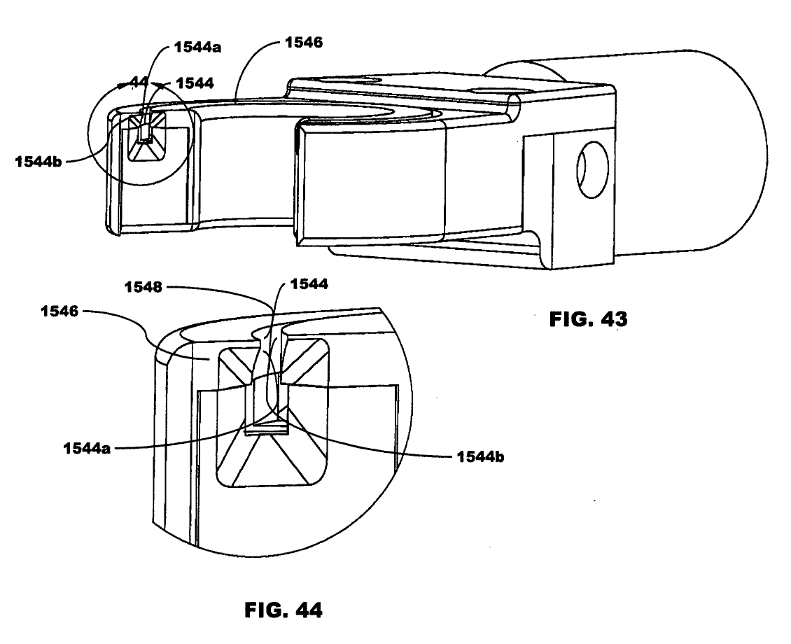

Turning to FIGS. 43 and 44 of the drawings, still another form of the cycling, suturing and knot-tying device of this invention is there illustrated. This embodiment is similar in most respects to the embodiment illustrated in FIGS. 33 through 38 and like numerals are used in FIGS. 43 and 44 to identify like components. The primary differences between this embodiment and that of FIGS. 33 through 38 resides in the somewhat differently configured opening 1544 formed in generally semi-circular-shaped body 1546 for receiving the chamfered end of the suturing needle as the needle is incrementally advanced. More particularly, opening 1544 is here specially configured in a manner to cause the chamfered end of the needle to be deflected by the strategically curved side walls 1544a and 1544b of the opening (FIG. 44) to be more precisely guided into and follow the curvature of the needle groove or guide of the shuttle member 1530 as the needle is incrementally advanced.

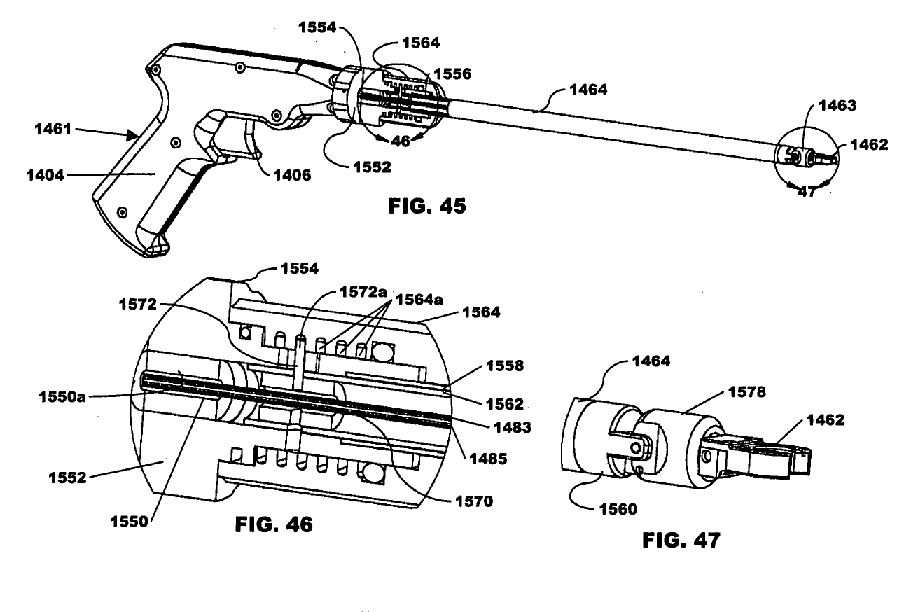

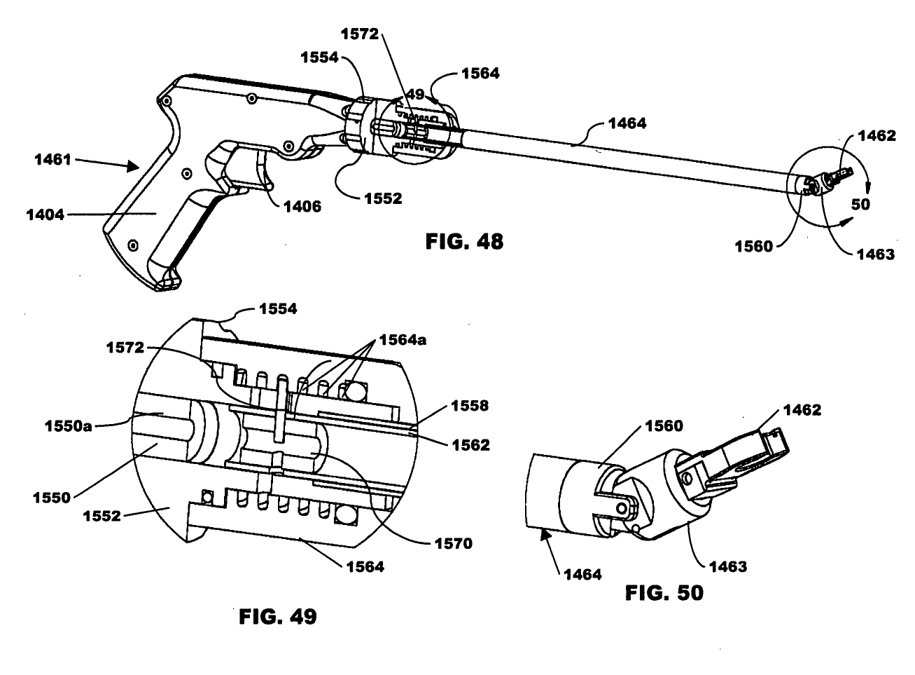

Referring now to FIGS. 45 through 50 as well as to FIGS. 22 through 24 of the drawings, the various mechanisms which cooperate to move the suture head 1462 of the device from a first angularly upward position to a second downward position relative to the barrel assembly 1464 are there illustrated. These novel mechanisms comprise the previously discussed suture head-positioning means of the invention for moving the suture head 1462 from a starting position shown in FIGS. 45 and 47 to a first angularly upward position shown in FIGS. 48 and 50 as well as to a second angularly downward position shown in FIGS. 51 and 53.

Turning particularly to FIGS. 22, 23 and 24, the previously identified barrel assembly 1464 can be seen to comprise a forwardly extending connector member 1550 that is interconnected with grip portion 1461 in the manner shown in FIG. 24. Connector member 1550 is provided with an axial bore 1550a through which cables 1483 and 1485 extend. Rotatably connected to grip portion 1461 and operably associated with connector member 1550 is a hub member 1552 that includes a finger-engaging knurled portion 1554 and a reduced-diameter portion 1556. Connected to reduced-diameter portion 1556 is an elongated outer tube 1558 to which a forward connector assembly 1560 is connected (FIG. 23).

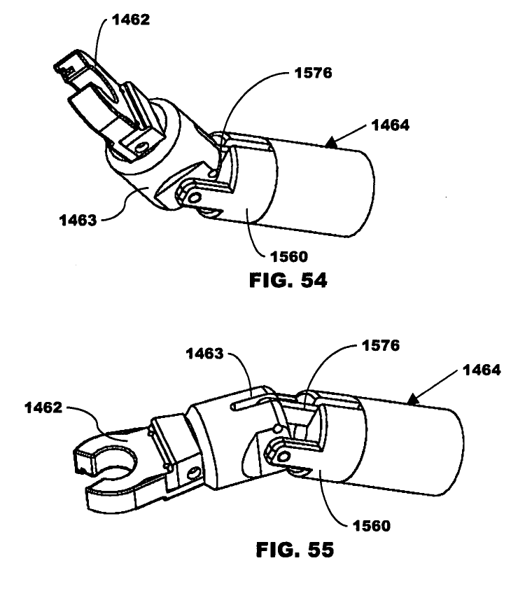

Operably associated with outer tube 1558 is the articulating-head operating means of the invention for moving the suture head 1462 of the device angularly upwardly and downwardly relative to the barrel assembly 1464 in the manner indicated in FIGS. 54 and 55. This novel articulating-head operating means here comprises an inner-tubular member 1562 which is reciprocally movable within outer tube 1558 by rotation of an internally threaded finger-gripping member 1564 that is rotatably carried by the reduced-diameter portion 1556 of hub 1552 (FIG. 24).

Connected proximate the forward end of inner-tubular member 1562 is a forward operating member 1568 and connected proximate the rearward end of inner-tubular member 1562 is a rearward operating member 1570 (FIGS. 23 and 24). Both forward operating member 1568 and rearward operating member 1570 include axial bores through which cables 1483 and 1485 extend. As best seen in FIGS. 24 and 46, rearward operating member 1570 also includes a radially outwardly extending drive rod 1572, the outer extremity 1572a of which operably engages the internal threads 1564a of finger-gripping member 1564. With this construction, rotation of finger-gripping member 1564 will cause the controlled reciprocal movement within outer tube 1558 between first and second positions of the inner-tubular assemblage made up of forward operating member 1568, inner-tubular member 1562 and rearward operating member 1570.

Pivotally connected to forward operating member 1568 is an elongated operating link 1576, the purpose of which will presently be described. As best seen in FIG. 23, the forward end 1576a of the operating link 1576 is pivotally connected to connector member 1463 (FIG. 45) to which the articulating-head 1462 is connected. As illustrated in FIGS. 48, 54 and 55 connector member 1463 is pivotally connected to forward connector assembly 1560 so that forward and rearward movement of the operating link 1576 relative to the forward connector assembly will cause the suture head 1462 of the device to move angularly upwardly and downwardly relative to the barrel assembly 1464.

With the construction described in the preceding paragraphs, when the operating components of the device are in the position shown in FIGS. 45, 46 and 47, rotation of finger-engaging knob 1564 in a clockwise direction to the position shown in FIG. 49 will cause the assemblage made up of forward operating member 1568, inner-tubular member 1562 and rearward operating member 1570 to move telescopically rearward within outer tube 1558. This rearward movement of the assemblage will cause the operating link 1576 to also move rearwardly in the manner shown in FIG. 54 resulting in the angularly upward movement of the suture head 1462 relative to the barrel assembly 1464 (see also FIGS. 48 and 50).

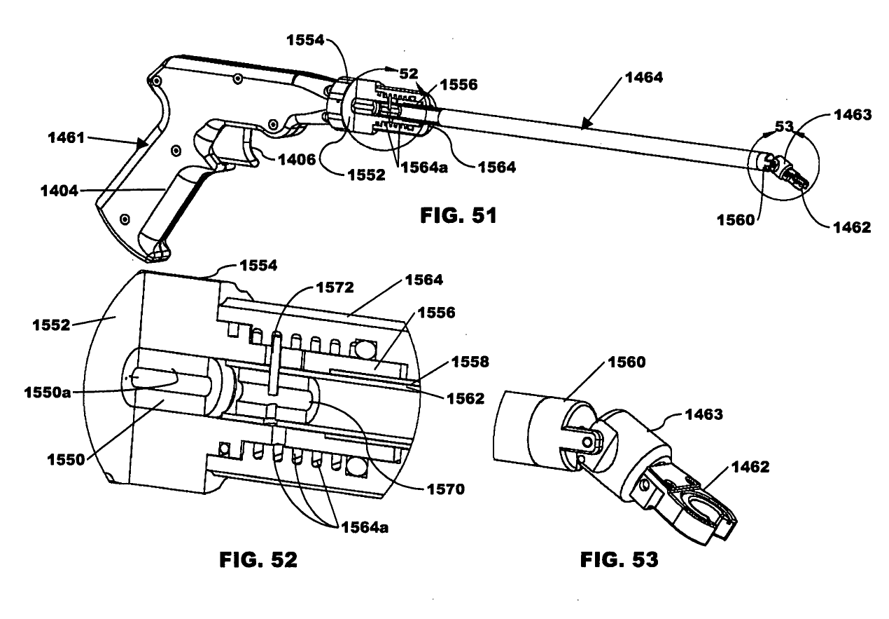

Similarly, with the operating components of the device in the position shown in FIGS. 45, 46 and 47, rotation of finger-engaging knob 1564 in a counterclockwise direction to the position shown in FIG. 52 will cause the assemblage made up of forward operating member 1568, inner-tubular member 1562 and rearward operating member 1570 to move telescopically forward within outer tube 1558. This forward movement of the assemblage will cause the operating link 1576 to also move forwardly in the manner shown in FIG. 55 resulting in the angularly downward movement of the suture head 1462 relative to the barrel assembly 1464 (see also FIGS. 51 and 53).

With the novel construction thus described, during surgery the physician need only slightly rotate the finger-engaging knob 1564 in either a clockwise or counterclockwise direction in order to angularly position the suture head 1462 of the device relative to the suture site. Similarly, by gripping knurled surface 1554 of hub 1552, the hub, along with the entire barrel assemblage, including the suture head 1462 can be controllably rotated relative to the suture site.

While the preferred embodiments of the invention have been described above, it will be recognized and understood that various modifications may be made in the invention and the appended claims are intended to cover all such modifications which may fall within the spirit and scope of the invention.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

We claim:

1. A suturing device comprising:
(a) a gripping portion;
(b) a head portion operably associated with said gripping portion, said head portion comprising:
(i) a body having a shuttle track;
(ii) a shuttle operably associated with said body for movement along said shuttle track between a first position and a second position, said shuttle being provided with a needle guide and at least one cavity;
(iii) a needle carried by said shuttle for movement along said needle guide between a first position and a second position; and
(iv) a needle-engaging member carried within said cavity for engagement with said needle to control movement of said needle along said needle guide; and
(c) operating means carried by said gripping portion for moving said shuttle between said first and second positions.

2. The suturing device as defined in claim 1, further including suture head-positioning means connected to said gripping portion for moving said suture head between said first angular position and said second angular position.

3. The suturing device as defined in claim 1 in which said device further includes a barrel portion connected to said gripping portion and in which said operating means comprises a trigger mechanism carried by said gripping portion and at least one operating cable carried by said barrel portion, said at least one operating cable having a first end connected to said shuttle and a second end connected to said trigger mechanism.

4. The suturing device as defined in claim 1 in which said needle-engaging member is movable within said cavity between first and second positions in response to movement of said shuttle between said first and second positions.

5. The suturing device as defined in claim 4 in which said shuttle further includes return means carried within said cavity for urging said needle-engaging member toward said first position.

6. The suturing device as defined in claim 4 in which said needle is semi-circular in shape and is provided with a multiplicity of circumferentially spaced-apart notches that are so constructed and arranged as to be engaged by said needle-engaging drive member as said needle-engaging member moves within said cavity.

7. The suturing device as defined in claim 4 in which said cavity has a rounded apex and angularly extending side walls and in which each said needle-engaging member comprises a rounded-head portion and a needle-engaging leg extending from said rounded-head portion.

8. The suturing device as defined in claim 4 in which said shuttle has a plurality of circumferentially spaced-apart cavities, each having a rounded portion and an open body portion and in which said suturing device includes a plurality of needle-engaging members, each said needle-engaging member having a rounded-head and a body portion extending from said rounded-head portion, said rounded-head portion being pivotally received within said rounded portion of said cavities and said body portion being constructed and arranged to engage said needle.

9. The suturing device as defined in claim 8 in which each of said circumferentially spaced-apart cavities is generally oval-shaped and includes opposing tapered side walls and in which each said needle-engaging member is generally round in cross section.

10. A suturing device comprising:
(a) a gripping portion comprising a handgrip and a trigger mechanism connected to said handgrip;
(b) an elongated, hollow-barrel portion connected to said gripping portion, said barrel portion comprising an elongated outer tube;
(c) an articulating-head portion connected to said barrel portion, said articulating-head portion comprising:
(i) a generally semi-circular-shaped body having a shuttle track;
(ii) a generally semi-circular-shaped shuttle operably associated with said generally semi-circular-shaped body for movement along said shuttle track from a first position to a second position and from said second position to a third and advanced position, said shuttle having first and second ends and being provided with a generally semi-circular-shaped needle guide and a plurality of circumferentially spaced cavities;
(iii) a generally semi-circular-shaped needle carried by said shuttle for movement along said needle guide between a first position and a second position, said needle having first and second ends, said first end being chamfered; and
(iv) a needle-engaging member carried within each of said plurality of circumferentially spaced cavities for engagement with said needle to control movement of said needle along said needle guide, said needle-engaging member being movable between a first position in engagement with said needle and a second position;
(d) operating means carried by said gripping portion and said barrel portion for controllably moving said shuttle, said operating means comprising: first and second operating cables carried by said hollow-barrel portion, said first operating cable having a first end connected to said first end of said shuttle and a second end connected to said trigger mechanism and said second operating cable having a first end connected to said second end of said shuttle and a second end connected to said trigger mechanism; and
(e) suture head-positioning means connected to said gripping portion for moving said suture head relative to said barrel between said first angular position and said second angular position.

11. The suturing device as defined in claim 10 in which said shuttle further includes return means carried within each of said circumferentially spaced-apart cavities for urging said needle-engaging member toward said first position.

12. The suturing device as defined in claim 10 in which said suture head-positioning means comprises:
(a) a hub member rotatably connected to said gripping portion, said hub member including a reduced-diameter portion connected to said elongated outer tube of said barrel portion;
(b) an inner-tubular assemblage carried by said elongated outer tube for controlled reciprocal movement therewithin between first and second positions;
(c) an internally threaded, finger-gripping member rotatably connected to said reduced-diameter portion of said hub and operably associated with said inner-tubular assemblage for moving said inner-tubular assemblage between first and second positions.

13. The suturing device as defined in claim 12 in which said suture head positioning means further comprises an operating link having a first end pivotally connected to said inner-tubular assemblage and a second end pivotally connected to said head-connector member.

14. The suturing device as defined in claim 13 in which said inner-tubular assemblage comprises inner-tubular member having first and second ends, a forward operating member connected to said first end and a rearward operating member connected to said second end.

15. The suturing device as defined in claim 14 in which said rearward operating member includes a radially outwardly extending drive rod that is operably associated with said internally threaded finger-gripping member.

16. A suturing device comprising:
(a) a gripping portion;
(b) an elongated, hollow-barrel portion connected to said gripping portion, said barrel portion comprising an elongated outer tube;
(c) an articulating-head portion connected to said barrel portion, said articulating-head portion comprising:
(i) a generally semi-circular-shaped body having a shuttle track;
(ii) a generally semi-circular-shaped shuttle operably associated with said generally semi-circular-shaped body for movement along said shuttle track from a first position to a second position, said shuttle having first and second ends and being provided with a generally semi-circular-shaped needle guide and a plurality of circumferentially spaced cavities;
(iii) a generally semi-circular-shaped needle carried by said shuttle for movement along said needle guide between a first position and a second position; and
(iv) a needle-engaging member carried within each of said plurality of circumferentially spaced cavities for engagement with said needle to control movement of said needle along said needle guide, said needle-engaging member being movable between a first position in engagement with said needle and a second position;
(d) operating means carried by said gripping portion and said barrel portion for controllably moving said shuttle; and
(e) suture-head-positioning means connected to said gripping portion for moving said suture head relative to said barrel between said first angular position and said second angular position, said suture-head-positioning means comprising:
(i) a hub member rotatably connected to said gripping portion, said hub member including a reduced-diameter portion connected to said elongated outer tube of said barrel portion;
(ii) an inner assemblage carried by said elongated outer tube for controlled reciprocal movement therewithin between first and second positions; and
(iii) an internally threaded, finger-gripping member rotatably connected to said reduced-diameter portion of said hub and operably associated with said inner-tubular assemblage for moving said inner-tubular assemblage between first and second positions.

17. The suturing device as defined in claim 16 in which said shuttle further includes return means carried within each of said circumferentially spaced-apart cavities for urging said needle-engaging member toward said first position.

18. The suturing device as defined in claim 16 in which said suture-head-positioning means further comprises an operating link having a first end pivotally connected to said inner-tubular assemblage and a second end pivotally connected to said head-connector member.

19. The suturing device as defined in claim 18 in which said inner assemblage comprises inner-tubular member having first and second ends, a forward operating member connected to said first end and a rearward operating member connected to said second end.

20. The suturing device as defined in claim 19 in which said rearward operating member includes a radially outward extending drive rod that is operably associated with said internally threaded finger-gripping member.
